SEQUENCE LISTING (16 sequences)

SEQ ID NO: 1 - DNA, 30 bp, Artificial Sequence
  primer
ttcatatgacacctcaaaatattactgatt

SEQ ID NO: 2 - DNA, 77 bp, Artificial Sequence
  primer
ttgccgcaattagtatggcaaatggtcctggaccacgtcgtaaacgctctgttggtcctgaaactctatgtggtgct

SEQ ID NO: 3 - DNA, 49 bp, Artificial Sequence
  primer
caataagaccaaagtctctagattacatacgataatttttgtttccagc

SEQ ID NO: 4 - DNA, 39 bp, Artificial Sequence
  primer
caataatctagattacatacgataatttttgtttccagc

SEQ ID NO: 5 - DNA, 31 bp, Artificial Sequence
  primer
cagcagccgcggtaatacagaggatgcaagc

SEQ ID NO: 6 - DNA, 31 bp, Artificial Sequence
  primer
ccgcgttgtttcatcaagccttacggtcacc

SEQ ID NO: 7 - DNA, 28 bp, Artificial Sequence
  primer
aggagcaataacgccctcttgataaaac

SEQ ID NO: 8 - DNA, 28 bp, Artificial Sequence
  primer
tgcacccctacctcctttatcactgagc

SEQ ID NO: 9 - DNA, 59 bp, Artificial Sequence
  primer
attttacatatgagacacatcaagatctggttccaaaaccgccgcatgaagtggaaaaa

SEQ ID NO: 10 - DNA, 47 bp, Artificial Sequence
  primer
ccgccgcatgaagtggaaaaagggtcctggaccacgtcgtaaacgat

SEQ ID NO: 11 - DNA, 31 bp, Artificial Sequence
  primer
gaattaaccgatcgacgtgctagcggacatt

SEQ ID NO: 12 - DNA, 315 bp, Homo sapiens
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatg

SEQ ID NO: 13 - DNA, 315 bp, Artificial Sequence
  engineered sequence
ggtcctgaaactctatgtggtgctgaattggtagacgctttacaattcgtttgtggcgatcgtggtttctacttcaacaaacctaccggttatggttctagctctcgtcgcgcaccacaaactggaattgtagatgagtgttgctttagaagttgtgatcttcgtcgccttgaaatgtactgtgctcctttgaaaccagccggttctgctcgtagtgttgcagctcaagctcataccgacatgcctaaaactcagaaggaagtacacttaaaaaatgcttcccgaggttctgctggaaacaaaaattatcgtatg

SEQ ID NO: 14 - DNA, 604 bp, Artificial Sequence
  plasmid construct
cataataataaaataaattaaatatgtcgaaatgttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattggg

SEQ ID NO: 15 - DNA, 207 bp, Artificial Sequence
  plasmid construct
gtcgactagtgtatagaaatccttttttttttatttttgcaaaccgcttttgatttacaaaaaattgaactagatccagatagatattggttgacacgggcatataagtcatgttatactgttaaataacaagcctttagttttccatttgaaaattcgtgcgcttgggagtccctgataattaaataaaccaagattttacatatg

SEQ ID NO: 16 - DNA, 649 bp, Artificial Sequence
  plasmid construct
gccaaatacatcattattgtatactctttcatatatatagcgcaacccaatttttcttttttactttttttttttttgaaatggaaatacctgactaacctaaataataagaacttcccccttgacagtggtatatgttgtatatgtaaatcctagatgtgaaaatatacggaattcctctatgaatctatgaaaggtataaaaaagaaagaaaaacacaaagaaaagaataggctaggcataaatcgatatgctgaaattgaaatcaaaaataaataagaagaggagccaattagatacaaataatgaatcgtaatagaaatagaaaataaagttcaggttcgaattccatagaatagataatatggatgggattgtctataatgatagacaaatgaaagactttctcaagattctgattcatccacttgagattttaaaattaaaataggttgggtgggcttgcaaattcactcagtctcagtgaataagtaaacaattgaatcggttcaattgcatggtgccaacgaaatcgagtgctaattcccattttattgaattaaccgatcgacgtgctagcggacatttattttgaattcgataatttttgcaaaaacatttcgacatatttatttattttattattatg

(19) United States
(12) Patent Application Publication
(10) Pub. No.: US 2021/0324396 A1
(43) Pub. Date: Oct. 21, 2021

(54) COMPOSITIONS AND METHODS FOR PRODUCTION OF ANTIBIOTIC FREE BIOPHARMACEUTICALS IN LETTUCE CHLOROPLASTS

(71) Applicant: THE TRUSTEES OF THE UNIVERSITY OF PENNSYLVANIA, Philadelphia, PA (US)

(72) Inventor: Henry Daniell, Media, PA (US)

(21) Appl. No.: 17/270,368

(22) PCT Filed: Aug. 22, 2019
(86) PCT No.: PCT/US2019/047721

§ 371 Date: Feb. 22, 2021

Related U.S. Application Data
(60) Provisional application No. 62/721,174, filed on Aug. 22, 2018.

(51) Int. Cl. C12N 15/82; C07K 14/65; A61P 19/00

(52) U.S. Cl. CPC C12N 15/8257; C12N 15/8214; A61K 38/00; A61P 19/00; C07K 14/65

(57) ABSTRACT

Compositions and methods for producing marker free biopharmaceutical proteins in the plastids of edible plants are disclosed. Also provided are methods for oral administration of such proteins to subjects in need thereof for the treatment of disease.

### CROSS REFERENCE TO RELATED APPLICATIONS

This application claims priority to U.S. Provisional Patent No. 62/721,174, filed Aug. 22, 2018, the entire disclosure being incorporated by reference as though set forth in full.

### STATEMENT OF FEDERALLY SPONSORED RESEARCH

This invention was made with government support under RO1 GM 63879, RO1 EY024564, RO1, HL 107904, RO1 HL 109442, RO1 133191 awarded by the National Institutes of Health. The government has certain rights in the invention.

### INCORPORATION BY REFERENCE OF MATERIAL SUBMITTED IN ELECTRONIC FORM

Applicant hereby incorporates by reference the Sequence Listing material filed in electronic form herewith. The file is labelled “6610WO00.txt”, was created Aug. 18, 2019, and is 5,881 bytes.

### FIELD OF THE INVENTION

This invention relates to production of biopharmaceuticals in higher plant chloroplasts. More specifically, the invention provides compositions and methods for production therapeutic proteins in the plastids of higher plants which lack selectable marker genes.

### BACKGROUND OF THE INVENTION

Several publications and patent documents are cited throughout the specification in order to describe the state of the art to which this invention pertains. Each of these citations is incorporated by reference herein as though set forth in full.

IGF-1 (Insulin-like growth factor-1) plays important roles upon binding to IGF-1 receptor in muscle formation, muscle mass and strength, regeneration after injury, and maintaining muscle health. IGF1 promotes skeletal muscle growth and recovery by regulating protein synthesis related to muscle fiber formation as well as by boosting proliferation of satellite cells on muscle fibers. After the satellite cells are differentiated, they fuse to damaged regions on the muscle fibers and repair or generate new fiber cells (Bikle et al., 2015). In fact, boosting IGF-1 levels in a rodent muscle model demonstrated functional improvement in muscle strength and limited fibrosis (Barton et al., 2002). Also, treatment of myotonic dystrophy type 1 patients with IGF-1/IGF-1 binding protein complex via subcutaneous injections for 24 weeks resulted in increased body mass and metabolism (Heatwole et al., 2011).

IGF-1 not only functions on formation of muscles but also activates bone remodeling and generation. Conditional deletion of IGF-1 results in reduced osteoblast number, activity, and bone mass in mice (Givoni et al., 2007). In contrast, overexpression of IGF-1 increased bone volume and remodeling (Jiang et al., 2006). Besides those, local delivery of IGF-1 accelerates bone formation in rat fracture healing model (Schmidmaier et al., 2002). Moreover, IGF-1 administration in patients increased bone healing, with rapid clinical improvements with hip or tibial fractures (Locatelli et al., 2014). In addition, IGF-1 facilitates survival, proliferation, differentiation in bone-resorbing osteoclasts and bone-forming osteoblasts. A previous study showed that decreased circulating IGF-1 resulted in reduction of bone density (Tahimic et al., 2013). IGF-1 also regulates tooth root development and root dentin stem cells. Exogenous IGF-1 stimulated proliferation and differentiation of isolated human tooth root stem cells (Wang et al., 2012). Aforementioned roles of IGF-1 in proliferation/differentiation of satellite cells and in muscle generation, together with bone growth/remodeling including dental root development can enhance its potential as a therapeutic for multiple muscle disorders as well as skeletal bone and periodontal diseases.

IGF-1 is present in three forms in the extracellular matrix: precursor IGF-1 which retains e-peptide (Pro-IGF-1), Pro-IGF-1 with N-glycosylation (Gly-Pro-IGF-1), and mature IGF-1 (Philippou et al., 2014). Due to its retention, potential biological activities of e-peptide have emerged for either the e-peptide alone or for the precursor. A previous study has shown that Pro-IGF-1 with e-peptide demonstrated positive effects for muscle disorders treatment and increased binding to the IGF-1R than mature IGF-1 whereas Gly-Pro-IGF-1 showed less efficiency at IGF-1R activation (Durzynska et al., 2013; Philippou and Barton, 2014). Current IGF-1 in clinical trials for muscle therapy is delivered via daily subcutaneous injections but lacking the e-peptide required for functional efficiency (clinicaltrials.gov/ct2/show/NCT01207908, Philippou and Barton, 2014). Orally deliverable IGF-1 in plant cells is preferable as patients avoid daily injections thereby increasing patient compliance, particularly treatments required for muscle disorders or bone healing, which are administered over long periods of time. Protein drugs expressed in transplastomic plants can be stored for several years at ambient temperature after lyophilization maintaining their efficacy (Su et al., 2015 and Herzog et al., 2017). Protection conferred by the cell wall from the digestive system enables oral delivery of such drugs. They can be delivered to target cells or tissues when gut bacteria lyse plant cell walls (Daniell et al., 2016 and Kwon et al., 2016). This natural process eliminates the need for prohibitively expensive fermentation, purification, and cold storage/transportation (Daniell et al., 2016a; Daniell et al., 2016b). Chloroplast genetic engineering has been developed and advanced in the past three decades to introduce valuable agronomic traits, expression of enzymes, biopharmaceuticals, or other high value products (Clarke et al., 2017; Daniell et al., 2016a; Daniell et al., 2016b; Jin and Daniell, 2015).

A significant drawback to the approaches described above is the presence of the antibiotic resistance gene (aadA) in transplastomic genomes which are needed for selection of the transplastomic lines, and to achieve homoplasmy (Daniell et al., 2016a; Jin and Daniell, 2015). These antibiotic resistance genes pose an important hurdle to advance these drugs in human clinical trials. This could be a challenge when thousands of copies of the aadA gene are present in each cell. Two different approaches have been used so far to remove selectable marker genes from transplastomic tobacco genomes. Use of direct repeats from flanking sequences has been used to loop out the aadA gene by the endogenous homologous recombination system present within chloroplasts (Day and Goldschmidt-Clermont, 2011). Alternatively, CRE (Corneille et al., 2001) or Bxb1 recombinase (Shao et al., 2014) and Lox or attP/attB sites have been used to remove the aadA gene from the small single copy region or the inverted repeat region of tobacco chloroplast genomes. However, use of Lox or attP/attB and recombinase complicates regulatory approval of marker gene excision process because of unintended excision or alteration of native genes due to non-homologous recombination in the nuclear genome, integration or use of exogenous recombinase and use of plant pathogens or pathogen sequences (viral promoters) for recombinase expression. In sharp contrast, chloroplast transformation process has been exempted from USDA-APHIS 7CFR part 340 regulations because of lack of use of plant pathogens or plant pathogen regulatory sequences. However, single-step marker gene removal has not yet been accomplished in edible crops expressing biopharmaceuticals via the chloroplast genome.

### SUMMARY OF THE INVENTION

In accordance with the present invention, a method for generating transplastomic plants producing antibiotic free biopharmaceuticals for oral consumption in plastids is provided. An exemplary method comprises introducing a plastid transformation vector into a plant cell, the vector comprising a selectable marker gene encoding an antibiotic, operably linked to a plastid promoter, the selectable marker gene and promoter being flanked by directly repeated DNA sequences between 500-800 nucleotides in length, said vector further comprising a heterologous nucleic acid comprising a second plastid promoter operably linked to a biopharmaceutical protein encoding sequence, wherein the protein of interest optionally includes a targeting peptide and, or, the nucleic acid encoding the protein is codon optimized for expression in said plant. Plant cells comprising the vector are then cultured in the presence of the antibiotic in a regeneration media for a suitable period for shoot production to occur. The shoots are assessed to confirm selectable marker gene excision and then transferred into antibiotic free media suitable for inducing root growth. Roots are then cultured to generate transplastomic plants expressing the protein of interest which lack the selectable marker gene and express the heterologous protein of interest in subsequent generations. In certain embodiments, the protein is IGF1 and glycosylation sites are removed from the protein coding sequence. In other embodiments, the protein of interest is purified from said plant.

The heterologous protein of interest, can include, without limitation, PTD-IGF1, CTB-IGF1, CTB-proinsulin, CTB-AMA, CTB-MSP, CTB-FIX, CTB-FIX-furin, CTB-Exendin, CTB-ESAT6, CTB-Mtb72F, CTB-VP1, CTB-MBP, CTB-Ace 2, CTB-Ang1-7, CTB-GAA, CTB-FVIII-HC, CTB-FVIII-C2, codon optimized CTB-FVIII-HC, codon optimized CTB-FVIII-LC, and codon optimized CTB-FVIII-SC. In a particularly preferred embodiment, the protein is selected from CTB-proIGF1 of PTD-proIGF1, each containing an e-peptide.

The plant can be any plant suitable for consumption and amenable to plastid transformation. In certain embodiments, the plant is selected from lettuce, tomato, carrot, low nicotine tobacco, or soybean. In certain embodiments, the plant is lettuce.

In yet another aspect, plant transformation vectors comprising the elements above are also within the scope of the invention.

Transplastomic plants comprising such vectors and producing biopharmaceutical proteins of interest generated using the methods described above are also provided.

In certain aspects, methods for treatment of subjects in need of IGF-1 therapy are also provided. An exemplary method entails oral administration of an effective amount of antibiotic free lettuce plants or lettuce plant products comprising IGF-1 protein operably linked to a PTD peptide and e-peptide which enhances IGF1 function, said administration being effective to alleviate or improve symptoms of genetic muscle diseases, acute atrophy, injuries or bone loss. While IGF-1 is exemplified herein, any of the proteins listed above can be expressed in the marker free plants described herein. Such plants and plant products would have a variety of utilities for the treatment of diabetes, coagulation disorders, inborn errors of metabolism disorders, etc.

### BRIEF DESCRIPTION OF THE DRAWINGS

FIGS. 1A-1G. Generation of lettuce/tobacco transplastomic lines expressing codon-optimized synthetic Pro-IGF-1 fused to native CTB. (FIG. 1A) Schematic illustration of CTB-Pro-IGF-1 expression cassette for lettuce chloroplast transformation. (FIG. 1A and FIG. 1E) 16s rRNA and 23s rRNA, 16s and 23s ribosomal RNA; trnI, isoleucyl-tRNA; trnA, alanyl-tRNA; Prrn, rRNA operon promoter; aadA, aminoglycoside 3′-adenylytransferase; TrbcL, 3′ UTR of ribulose bisphosphate carboxylase large subunit; PpsbA, promoter of psbA; CTB-Pro-IGF-1(co), codon optimized premature form of human insulin-like growth factor-1 with CTB (Cholera non-toxic B subunit) fusion; TpsbA, 3′-UTR of psbA; SB-P, Southern blot probe. Primer sets used for PCR screening are indicated in arrows. (FIG. 1B) Genomic DNA PCR screening and (FIG. 1C) Southern blot analysis, and (FIG. 1D) protein expression of the lettuce lines expressing CTB-Pro-IGF-1. Expected sizes are indicated in arrows (24.3 kDa). (FIG. 1B-FIG. 1D) Lanes 1 to 4, individual transplastomic lines; WT, untransformed wild type; LS, lettuce. (FIG. 1E) Schematic illustration of CTB-Pro-IGF-1 expression cassette for tobacco chloroplast transformation. (FIG. 1F) Southern blot analysis of CTB-Pro-IGF1 tobacco transplastomic lines. Lanes 1 and 2, individual wild type plants or transplastomic lines; WT, untransformed wild type; PH, tobacco Petit Havana. (FIG. 1G) Sequence Alignments of Native (N; SEQ ID NO: 12) and Codon-Optimized (C; SEQ ID NO: 13) Pro-IGF-1. Optimized codons are marked in yellow. Nat: native sequence; CH: codon-optimized sequence. To avoid glycosylation, Lsy68 (AAG), Arg74 (CGT) and Arg77 (CGC) were changed to Gly68 (GGT), Ala74 (GCA), and Ala77 (GCT) respectively, which are marked in red.

FIGS. 2A-2J. Generation of lettuce marker-free transplastomic lines expressing codon-optimized PTD-Pro-IGF-I. (FIG. 2A) Schematic diagram of chloroplast transformation vector containing PTD-Pro-IGF-1 expression cassette and marker excision process is shown. 16s rRNA and 23s rRNA, 16s and 23s ribosomal RNA; trnI, isoleucyl-tRNA; atpB; chloroplast encoded CF1 ATP synthase subunit beta; Prrn, rRNA operon promoter; aadA, aminoglycoside 3′-adenylytransferase; TrbcL, 3′ UTR of ribulose bisphosphate carboxylase large subunit; PpsbA, promoter of psbA; PTD-Pro-IGF-1(co), codon optimized premature form of human insulin-like growth factor-1 with PTD (Protein Transduction Domains) fusion; TpsbA, 3′-UTR of psbA; trnA, alanyl-tRNA; SB-P, Southern blot probe. (FIG. 2B) Genomic DNA PCR screening, (FIG. 2C) Southern blot, and (FIG. 2D) protein expression of PTD-Pro-IGF-1 lines. Expected size is indicated in an arrow (14.5 kDa) (FIGS. 2B-2D) Lanes 1 to 4, four individual T0 transplastomic lines; WT, untransformed wild type; MF, lettuce marker-free. Expected sizes are indicated in arrows. (FIG. 2E) PCR screening, (FIG. 2F) Southern blot, and (FIG. 2G) protein expression of PTD-Pro-IGF-1 in antibiotic-free T1 generation. (FIG. 2E and FIG. 2F) Lanes 1 to 10; ten individual T1 transplastomic plants, WT; untransformed wild type lettuce; PC, positive control with marker. Expected sizes are indicated in arrows (3.3/2.4/2.4 kb of PCR products and 14.5 kDa of PTD-Pro-IGF-1). (FIG. 2H) Southern blot and (FIG. 2I) protein expression of PTD-Pro-IGF-1 in antibiotic-free T2 generation. (FIG. 2H and FIG. 2I) Lanes 1 to 4; four individual T2 transplastomic plants, WT; untransformed wild type lettuce. Expected sizes are indicated in arrows (14.5 kDa). (FIG. 2J) Schematic representation of cloning process to construct a marker free lettuce chloroplast transformation vector (pLsLF-MF). Two repeated DNA sequences (649 bp, atpB) were flanked to aadA expression cassette as shown in the diagram. PTD-Pro-IGF-1 DNA sequence was inserted between PpsbA and TpsbA for expression in chloroplasts. 16s rRNA and 23s rRNA, 16s and 23s ribosomal RNA; trnI, isoleucyl-tRNA; atpB; chloroplast encoded CF1 ATP synthase subunit beta; Prrn, rRNA operon promoter; aadA, aminoglycoside 3′-adenylytransferase; TrbcL, 3′ UTR of ribulose bisphosphate carboxylase large subunit; PpsbA, promoter of psbA; PTD-Pro-IGF-1(co), codon optimized premature form of human insulin-like growth factor-1 with PTD (Protein Transduction Domains) fusion; TpsbA, 3″-UTR of psbA; trnA, alanyl-tRNA.

FIGS. 3A-3P. Characterizations of CTB/PTD-Pro-IGF-1 in lettuce/tobacco transplastomic lines after lyophilization. (FIG. 3A-FIG. 3D) Immunoblot assays of CTB-Pro-IGF-1 expressed in (FIG. 3A and FIG. 3B) lettuce and (FIG. 3C and FIG. 3D) tobacco lyophilized cells using (FIG. 3A and FIG. 3C) anti-CTB and (FIG. 3B and FIG. 3D) anti-IGF-1 antibodies. (FIG. 3E) Quantification of PTD-Pro-IGF-1 expressed in lyophilized marker-free lettuce cells. (FIG. 3F and FIG. 3G) Comparison of CTB/PTD-Pro-IGF-1 expressions between equal amount (1×) of fresh and lyophilized leaf materials. (FIG. 3F) Lettuce CTB-Pro-IGF-1 and (FIG. 3G) marker-free lettuce PTD-Pro-IGF-1 transplastomic lines. RbcL was used as a loading control. (FIG. 3A-FIG. 3G, FIG. 3K and FIG. 3L) The expected sizes of CTB-Pro-IGF-1 and PTD-Pro-IGF-1 at 24.3 kDa and 14.5 kDa, respectively, are indicated in arrows. PH, tobacco Petit Havana; LS, lettuce; MF, marker-free lettuce; WT, untransformed wild type tobacco or lettuce. CTB or IGF-1 peptide were used as controls. (FIG. 3H and FIG. 3I) ELISA of CTB-Pro-IGF-1 pentamer form in (FIG. 3H) lettuce and (FIG. 3I) tobacco for CTB-GM1 receptor binding. Data are representative of two biological repeats run in triplets. Data are expressed as the mean±SEM (***P-value<0.001 vs. wild type by ANOVA). CTB was used as a positive control. Lyo, lyophilized leaves. (FIG. 3J) Nonreducing immunoblot analysis of tobacco expressing CTB-Pro-IGF-1 using anti-CTB antibody. (FIG. 3K and FIG. 3L) Comparison of (FIG. 3K) CTBPro-IGF-1 and (FIG. 3L) PTD-Pro-IGF-1 expressions between T0 and T1 generations. RbcL was used as a loading control. Data are expressed as the mean±SEM (**P-value≤0.01 by ANOVA). (FIG. 3M-FIG. 3N) Expression levels of (FIG. 3M) CTB-Pro-IGF-1 and (FIG. 3N) PTD-Pro-IGF-1 in lyophilized lettuce at the indicated time points of storage duration using western blot assay. Data are expressed as the mean±SEM (P-value≥0.5 by ANOVA between groups). (FIG. 3O) Germination of marker removed PTD-Pro-IGF-1 lettuce lines. Five seeds of PTD-Pro-IGF-1 and five seeds of wild type lettuce were germinated on spectinomycin containing media. The pictures were taken on day 6 (top) and day 15 (bottom) after germination respectively. Spec25, 25 μg/mL of spectinomycin; Spec50, 50 μg/mL of spectinomycin. (FIG. 3P) Purified CTB-Pro-IGF-1 from lyophilized plant cells. Total protein, total soluble protein extracted from CTB-Pro-IGF-1 expressed lyophilized plant cells before the purification; Co, Coomassie blue staining; W, western blot against anti-CTB. The arrows indicate approximately 24.3 kDa of monomeric CTB-Pro-IGF-1.

FIGS. 4A-4E. CTB-Pro-IGF-1 promotes keratinocyte, GMSCs and osteoblast cell proliferation and osteoblast differentiation. (FIG. 4A-FIG. 4D) Relative absorbance of viable (FIG. 4A) HOK (Human Oral Keratinocytes), (FIG. 4B) GMSCs (Human Gingiva derived Mesenchymal Stromal Cells), (FIG. 4C) SCCs (Human head and neck Squamous Carcinoma Cells), and (FIG. 4D) MC3T3 (Mouse Osteoblast Cells) after incubation with purified CTB-Pro-IGF-1. IGF-1 peptide was utilized as positive control. Data are representative of two biological repeats run in triplets. Data are expressed as the mean±SEM. * indicates P-value<0.05 vs. IGF-1 by ANOVA. (FIG. 4E) qPCR analysis of osteogenesis marker genes: Alkaline phosphatase (ALP), runt related transcription factor 2 (RUNX2), and Osterix (Osx). Mouse osteoblastic cell line MC3T3 #4 cells was induced with osteogenic medium (OS, DMEM, FBS 10%, 50 μg/ml ascorbic acid and 10 mM β-glicerophosphate) and purified CTB-Pro-IGF-1 at 100 ng/ml, 200 ng/ml and 300 ng/ml, respectively for 5 days. *, P<0.05 vs. CON; #, P<0.05 vs. OS.

FIGS. 5A-5C. Delivery of IGF-1 following oral gavage. (FIG. 5A) Absorption of IGF-1 in sera at 2 hours after oral gavage. C57BL/6J mice (n=14 to 16 mice per group) were fed a 5 μg dose of CTB/PTD-IGF-1 expressed in lyophilized cells. Data points represent individual male (blue dots) or female (pink dots) mice. ** indicates P-value≤0.01 compared to the baselines. (FIG. 5B) Increased IGF-1 in gastrocnemius muscle at 5 hours after oral administration of CTB-Pro-IGF-1 (left) and densitometric quantitation using western blots, with GAPDH as an internal loading control (right). Data are expressed as the Mean±SD. (FIG. 5C) Increased IGF-1 in soleus muscle at 5 hours after oral administration of PTD-Pro-IGF-1 by immunoblot against anti-IGF-1 (left), after normalization to β-actin (right). Data are expressed as the mean±SD. ** indicates P-value<0.01 vs. unfed by ANOVA.

FIGS. 6A-6B. Administration of CTB-Pro-IGF-1 by oral gavage promotes bone regeneration in diabetic fracture healing. (FIG. 6A) Representative images of 3-D reconstructs from sections of micro CT scan of the fracture site at 6 weeks post fracture and quantitative measurements of the newly formed bone at the fracture gap. (FIG. 6B) Histological analyses and relative bone are for each group. Data are expressed as the mean±SEM. * P<0.05 between groups. ** P<0.01 between groups. Non-Dia WT group (nondiabetic mice, oral gavage with lyophilized wide type plant cells), Non-Dia IGF group (non-diabetic mice, oral gavage with lyophilized plant cells containing CTB-Pro-IGF-1), Dia WT group (diabetic mice, oral gavage with lyophilized wide type plant cells), Dia IGF group (diabetic mice, oral gavage with lyophilized plant cells containing CTB-Pro-IGF-1).

FIGS. 7A-7B. Cloning strategy for construction of plant chloroplast vectors for expression of antibiotic free biopharmaceuticals. (FIG. 7A) aadA based transformation was used to duplicate a 649 bp region of plastid DNA corresponding to the atpB promoter region. Homology-based excision ensures complete removal of functional aadA genes. (SEQ ID NO: 14 on left and SEQ ID NO: 15 on right) (FIG. 7B) shows the final vector construct. (FIG. 7C) provides the sequence of the 649 base pair repeat (SEQ ID NO: 16). The native atpB repeat sequence (649 bp) contains rbcL and atpB promoter regions in opposite directions and this disrupts gene expression by making antisense RNA when transgene cassette is introduced into the transcriptionally active spacer regions. However, such spacer regions are preferred because transgene expression level is higher than transcriptionally silent spacer regions (Jin & Daniell, Trends in Plant Science, 2015; Daniell et al, Genome Biology 2016). Therefore, one or both promoter regions were deleted in different versions of the marker-free expression cassette.

FIG. 8 is a table showing the frequency of codon usage of psbA, native Pro-IGF-1, and codon-optimized Pro-IGF-1.

### DETAILED DESCRIPTION OF THE INVENTION

Human insulin-like growth factor-1 (IGF-1) plays important roles in development and regeneration of skeletal muscle and bones but requires daily injections or surgical implantation in dental applications. Current clinical IGF-1 lacks e-peptide and is glycosylated, reducing functional efficacy. Although previous studies reported IGF-1 expression in seeds, non-edible plants, or cell cultures, Pro-IGF-1 retaining e-peptide has never been expressed or functionally evaluated in suitable animal models following oral delivery.

As described herein, Pro-IGF-1 with e-peptide (fused to cell penetrating peptides) was codon optimized and expressed in lettuce chloroplasts, without the antibiotic resistance gene. The antibiotic resistance (aadA) gene was quickly excised via homologous recombination of direct repeats and marker-free chloroplast genomes were maintained in lettuce plants in the next generation, maintained high level Pro-IGF-1 expression; folding, assembly, stability and functionality was maintained in lyophilized plant cells at ambient temperature for several months/years. CTB-Pro-IGF-1 stimulated growth of cultured human oral keratinocytes, gingiva-derived mesenchymal stromal cells and mouse osteoblasts in a dose-dependent manner and upregulated the expression of osteoblast marker genes including ALP, OSX, and RUNX2. Mice orally gavaged with freeze-dried plant cells significantly increased IGF-1 in sera and muscle in male and female mice with equal efficiency and promoted bone regeneration in a diabetic fracture healing model. Biopharmaceutical expression in plant cells without antibiotic resistance gene for the first time, with long-term storage at ambient temperature and the convenience of repetitive oral delivery should enhance affordability and patient compliance by eliminating daily injections or surgical implantations.

### Definitions

It is to be understood that both the foregoing general description and the following detailed description are exemplary and explanatory only and are not intended to limit the scope of the current teachings. In this application, the use of the singular includes the plural unless specifically stated otherwise. For example, “at least one” means that more than one can be present. Also, the use of “comprise”, “contain”, and “include”, or modifications of those root words, for example but not limited to, “comprises”, “contained”, and “including”, are not intended to be limiting and means “including the following elements but not excluding others.” The term “consists essentially of,” or “consisting essentially of,” as used herein, excludes other elements from having any essential significance to the combination. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “disease,” “disorder,” or “complication” refers to any deviation from a normal state in a subject.

An “antibiotic-free biopharmaceutical” as used herein refers to a biopharmaceutical produced in the chloroplasts of higher plants which lack a selectable marker gene encoding an antibiotic.

As used herein, by the term “effective amount” “amount effective,” or the like, it is meant an amount effective at dosages and for periods of time necessary to achieve the desired result.

As used herein, the term “inhibiting” or “treating” means causing the clinical symptoms of the disease state not to worsen or develop, e.g., inhibiting the onset of disease, in a subject that may be exposed to or predisposed to the disease state, but does not yet experience or display symptoms of the disease state.

As used herein, the term “CTB” refers cholera toxin B subunit. Cholera toxin is a protein complex comprising one A subunit and five B subunits. The B subunit is nontoxic and important to the protein complex as it allows the protein to bind to cellular surfaces via the pentasaccharide chain of ganglioside.

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “promoter region” refers to the 5′ regulatory regions of a gene (e.g., 5′UTR sequences (e.g., psbA sequences, promoters (e.g., universal Prnn promoters or psbA promoters endogenous to the plants to be transformed and optional enhancer elements.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The term “selectable marker gene” refers to a gene that when expressed confers a selectable phenotype, such as antibiotic resistance, on a transformed cell or plant. Selectable markers useful in plastid transformation vectors include, without limitation, those encoding for spectinomycin resistance, glyphosate resistance, BADH resistance, and kanamycin resistance.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. However, most preferred for use in the invention are plastid transformation vectors. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

As used herein, the term “chloroplast” includes organelles or plastids found in plant cells and other eukaryotic organisms that conduct photosynthesis. Chloroplasts capture light energy to conserve free energy in the form of ATP and reduce NADP to NADPH through a complex set of processes called photosynthesis. Chloroplasts contain chlorophyll. Chloroplasts have a higher copy number and expression levels of the transgene. Each chloroplast may contain up to 100 genomes, while each plant cell may contain up to 100 chloroplasts. Therefore, each plant cell may contain as many as 100000 chloroplast genomes which results in high expression levels of proteins expressed via the chloroplast genome. Chloroplasts further offer gene containment through maternal inheritance as the chloroplast genome is not transferred through pollen unlike nuclear genomic DNA. Chloroplasts have the ability to transcribe polycistronic RNA and can perform the correct processing of eukaryotic proteins including the ability to carry out posttranslational modifications such as disulphide bonding, assembly of multimers and lipid modifications.

As used herein, a “composition,” “pharmaceutical composition” or “therapeutic agent” all include a composition comprising an IGF-1 comprising construct as described herein. Optionally, the “composition,” “pharmaceutical composition” or “therapeutic agent” further comprises pharmaceutically acceptable diluents or carriers.

As used herein, the term “expression” in the context of a gene or polynucleotide involves the transcription of the gene or polynucleotide into RNA. The term can also, but not necessarily, involves the subsequent translation of the RNA into polypeptide chains and their assembly into proteins.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to an administrable composition for treating or preventing disease via administration of a therapeutic fusion protein produced in a plant chloroplast. The composition comprises a therapeutically-effective amount of the fusion protein expressed by a plant and a plant remnant.

Proteins expressed in accord with certain embodiments taught herein may be used in vivo by administration to a subject, human or animal in a variety of ways. The pharmaceutical compositions may be administered orally or parenterally, i.e., subcutaneously, intramuscularly or intravenously, though oral administration is preferred.

Oral compositions produced by embodiments of the present invention can be administered by the consumption of the foodstuff that has been manufactured with the transgenic plant producing the plastid derived therapeutic fusion protein. The edible part of the plant, or portion thereof, is used as a dietary component. The therapeutic compositions can be formulated in a classical manner using solid or liquid vehicles, diluents and additives appropriate to the desired mode of administration. Orally, the composition can be administered in the form of tablets, capsules, granules, powders, chewable gums, and the like with at least one vehicle, e.g., starch, calcium carbonate, sucrose, lactose, gelatin, etc. The preparation may also be emulsified. The active immunogenic or therapeutic ingredient is often mixed with excipients which are pharmaceutically acceptable and compatible with the active ingredient. Suitable excipients are, e.g., water, saline, dextrose, glycerol, ethanol or the like and combination thereof. In addition, if desired, the compositions may contain minor amounts of auxiliary substances such as wetting or emulsifying agents, pH buffering agents, or adjuvants. In a preferred embodiment the edible plant, juice, grain, leaves, tubers, stems, seeds, roots or other plant parts of the pharmaceutical producing transgenic plant is ingested by a human or an animal thus providing a very inexpensive means of treatment of or immunization against disease.

In a specific embodiment, plant material (e.g. lettuce, tomato, carrot, soybean, low nicotine tobacco material etc) comprising chloroplasts capable of expressing the therapeutic fusion protein, is homogenized and encapsulated. In one specific embodiment, an extract of the lettuce material is encapsulated. In an alternative embodiment, the lettuce material is powderized before encapsulation.

In alternative embodiments, the compositions may be provided with the juice of the transgenic plants for the convenience of administration. For said purpose, the plants to be transformed are preferably selected from the edible plants consisting of tomato, carrot and apple, among others, which are consumed usually in the form of juice.

According to another embodiment, the subject invention pertains to a transformed chloroplast genome that has been transformed with a vector comprising a heterologous gene that expresses a therapeutic fusion protein or peptide as disclosed herein.

Reference to the protein sequences herein relate to the known full length amino acid sequences as well as at least 12, 15, 25, 50, 75, 100, 125, 150, 175, 200, 225, 250 or 265 contiguous amino acids selected from such amino acid sequences, or biologically active variants thereof. Typically, the polypeptide sequences relate to the known human versions of the sequences.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active therapeutic fusion polypeptide can readily be determined by assaying for native activity, as described for example, in the specific Examples, below.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% base pair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentrations should be chosen that is approximately 12-20° C. below the calculated Tₘ of the hybrid under study. The Tₘ. of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962): Tm=81.5° C.−16.6(log 10 [Na+])+0.41(% G+C)−0.63(% formamide)−600/l), where l=the length of the hybrid in base pairs. Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. The following materials and methods are provided to facilitate the practice of the present invention.

### Codon Optimization

To maximize the expression of heterologous genes in chloroplasts, a chloroplast codon optimizer program was developed based on the codon preference of psbA genes across 133 seed plant species. All sequences were downloaded from the National Center for Biotechnology Information (NCBI, ncbi.nlm.nih.gov/genomes/GenomesGroup.cgi?taxid=2759&opt=plastid).

The usage preference among synonymous codons for each amino acid was determined by analyzing a total of 46,500 codons from 133 psbA genes. The optimization algorithm (Chloroplast Optimizer v2.1) was made to facilitate changes from rare codons to codons that are frequently used in chloroplasts using JAVA.

### Marker Free Edible Plants

As described above, a method for generating edible transplastomic plants producing antibiotic free biopharmaceuticals for oral consumption in plastids is provided. A number of exemplary plastid transformation vectors for this purpose are provided in the figures. In certain embodiments, once the method is performed, the resulting plants are assessed for homoplasmy. As exemplified herein, the selectable marker gene can be an aadA gene and the antibiotic can be spectinomycin. While aadA and spectinomycin resistance are exemplified herein, other antibiotic encoding selectable marker genes and antibiotics could also be used in the compositions and methods of the invention.

The native atpB repeat sequence (649 bp) or variants thereof used in the vectors described contains rbcL and atpB promoter regions in opposite directions and this disrupts gene expression by making antisense RNA when a transgene cassette is introduced into the transcriptionally active spacer regions. However, such spacer regions are preferred because transgene expression level is higher than transcriptionally silent spacer regions (Jin & Daniell, Trends in Plant Science, 2015; Daniell et al, Genome Biology 2016). Therefore, at least one or both promoter regions were deleted in the repeat sequences in different versions of the marker-free expression cassette.

In certain embodiments, the direct repeat sequences in the vectors are encoded by SEQ ID NO: 16. In other embodiments, the direct repeats are encoded by SEQ ID NO: 16, where one, two, three, four or all of the sequences shown in bold, underlined or in blue or red are deleted.

The biopharmaceutical proteins of the invention can be encoded as fusion proteins. In certain aspects, a PTD peptide will be operably linked to the protein of interest. In certain other aspects a CTB peptide will be employed

The successful production of marker free IGF-1 producing plants paves the way for production of additional therapeutically beneficial proteins, including without limitation, CTB-proinsulin, CTB-AMA, CTB-MSP, CTB-FIX, CTB-FIX-furin, CTB-Exendin, CTB-ESAT6, CTB-Mtb72F, CTB-VP1, CTB-MBP, CTB-Ace 2, CTB-Ang1-7, CTB-GAA, CTB-FVIII-HC, CTB-FVIII-C2, codon optimized CTB-FVIII-HC, codon optimized CTB-FVIII-LC, and codon optimized CTB-FVIII-SC.

The following materials and methods are provided to facilitate the practice of the present invention.

Generation of CTB-Pro-IGF-1 Expressing Tobacco and Lettuce Transplastomic lines

Codon-optimized and synthesized Pro-IGF-1 described above was overlap PCR amplified with NdeI-CTB-F, 5′-TTCATATGACACCTCAAAATATTACTGATT (SEQ ID NO: 1); CTB-IGF-1, 5′-TTGCCGCAATTAG TATGGCAAATGGTCCTGGACCACGT CGTAAACGCTCTGTTGGTCCTGAAACTCTATGTGGTGCT (SEQ ID NO: 2); IGF-1-PshAI-R, 5′-CAATAAGACCAAAGTCTCTAGATTACATACGATAATT TTTGTTTCCAGC (SEQ ID NO:3); and IGF-1-XbaI-R, 5′-CAATAATCTAGATTACATACGATAATTTTTGTTTCCAGC (SEQ ID NO: 4). The amplified CTB-Pro-IGF-1 was cloned into tobacco chloroplast transformation vector (pLD-utr) and lettuce vector (pLsLF) (Kwon et al., 2017; Verma et al., 2008). The cloned CTB-Pro-IGF-1 was transformed to tobacco (Petit Havana) and Lettuce (Lactuca sativa) cv. Simpson Elite by bombardment as previously described (Kwon et al., 2017; Verma et al., 2008). After the bombardment, regeneration was induced as described previously (Verma et al., 2008). Primary shoots were PCR screened with 16s-F, 5′-CAGCAGCCGCGGTAATACAGAGGATGCAAGC (SEQ ID NO: 5); aadA-R, 5′-CCGCGTTGTTTCATCAAGCCTTACGGTCACC (SEQ ID NO: 6); NdeI-CTB-F, 5′-TTCATATGACACCTCAAAATATTACTGATT (SEQ ID NO: 1); IGF-1-PshAI-R, 5′-CAATAAGACCAAA GTCTCTAGATTACATACGATAATTTTTGTTTCCAGC (SEQ ID NO:3); UTR-F, 5′-AGGAGCAATAACGCCCTCTTGATAAAA C (SEQ ID NO: 7); and 23s-R, 5′-TGCACCCCTACCTCCTTTATCACTGAGC (SEQ ID NO: 8). The PCR positive leaves were induced for the 2^{nd} round regeneration on regeneration media (Verma et al., 2008) containing spectinomycin 50 μg/mL. Secondary shoots were screened by the 2^{nd} round PCR with the same primer sets used in the 1^{st} round selection. Screened positive shoots were induced to form roots. After they were confirmed to be homoplasmic and protein expression was confirmed by Southern blots and western blots, respectively, as described below, they were transferred to hydroponic system and greenhouse as described previously (Kwon et al., 2017).

### Creation of Marker-Free PTD-IGF-1 Lettuce Chloroplast Expression Vectors

Previously made lettuce chloroplast transformation vector in Henry Daniell's lab, pLsLF, was used as a template (Daniell et al., 2016a), which contains spectinomycin-resistant gene (aadA, aminoglycoside 3′-adenylytransferase gene). To excise the marker gene, directly repeated DNA sequence (PatpB and 5′ UTR, 649 bp (Kode et al., 2006)) was PCR amplified using tobacco total genomic DNA as a template and then the sequence-confirmed direct repeats were cloned to flank aadA expression cassette. To validate the operation of marker-free vector after transformation of chloroplasts, ptxD (phosphite oxidoreductase, GenBank: AAC71709.1) gene from Pseudomonas stutzeri was cloned under the control of psbA promoter. To facilitate the selection of marker-free transformants on phosphite media, codon optimized ptxD was also tested along with native sequence of ptxD (data not shown). For the insertion of single-digested atpB fragments into vector backbone, NEBuilder HiFi DNA (NEB, Ipswich, Mass.) assembly kit was used to avoid the possible ligation of the fragments in a reverse direction. After codon-optimization as previously described (Kwon et al., 2016), Pro-IGF-1 was synthesized in GenScript (Piscataway, N.J.). The Pro-IGF-1 was overlap PCR amplified with NdeI-PTD-F, 5′-ATTTTACATATGAGACACATCAAGATCTGGTTCCAAAACCGCCGCATGAAGTGGAA AAA (SEQ ID NO: 9); PTD-IGF-1-F, 5′-CCGCCGCATGAAGTGGAAAAA GGGTCCTGGACCACGTCGTAAACGAT (SEQ ID NO: 10); and IGF-1-PshAI-R, 5′-CAATAAGACCAAAGTCTCTAGATTACA TACGATAATTTTTGTTTCCAGC (SEQ ID NO: 3). PTD fused Pro-IGF-1 was replaced with ptxD in the pLsLF-MF vector between NdeI and PshAI sites for chloroplast transformation. Lettuce (Lactuca sativa) cv. Simpson Elite leaves were bombarded for transformation as previously described (Verma et al., 2008).

### Screening of Antibiotic Gene Deleted Lettuce Containing PTD-Pro-IGF-1

After bombardment, lettuce leaves were cut into small pieces (less than 1 cm²) and grown on regeneration media [MS salts (Caisson, Smithfield, Utah), Gamborg vitamins (PhytoTechnology Laboratory, Lenexa Kans.), BAP 0.2 mg (Sigma, St Louis, Mo.), NAA 0.1 mg (Sigma, St Louis, Mo.), Myoinositol 100 mg (Sigma, St Louis, Mo.), PVP 500 mg (Sigma, St Louis, Mo.), sucrose 30 g (Sigma, St Louis, Mo.), phytablend 5 g (PhytoTechnology Laboratory, Lenexa, Kans.)] containing spectinomycin 50 μg/mL. Generated primary shoots (4-6 weeks) were screened using specific PCR primer sets

(16s-F,
(SEQ ID NO: 5)
5′-CAGCAGCCGCGGTAATACAGAGGATGCAAGC;
aadA-R,
(SEQ ID NO: 6)
5′-CCGCGTTGTTTCATCAAGCCTTACGGTCACC;
atpB-R,
(SEQ ID NO: 11)
5′-GAATTAACCGATCGACGTGCTAGCGGACATT;
UTR-F,
(SEQ ID NO: 7)
5′-AGGAGCAATAACGCCCTCTTGATAAAAC;
23s-R,
(SEQ ID NO: 8)
5′-TGCACCCCTACCTCCTTTATCACTGAGC.

Positive shoots were regenerated for another round on spectinomycin at 50 μg/mL. As soon as shoots started to bleach out, they were assessed by PCR for aadA gene excision, and transferred to spectinomycin-free media to induce roots. Once the roots were generated, transgene expression and homoplasmy were confirmed by western blots and Southern blots respectively. For the western blots, after fresh leaves were ground in liquid nitrogen, 100 mg of the ground powder was suspended in 300 μL of extraction buffer (100 mM NaCl, 10 mM EDTA, 200 m Tris-Cl, pH 8, 0.05% v/v Tween 20, 0.1% v/v SDS, 400 mM Sucrose, 14 mM β-mercaptoethanol, 100 mM DTT, 2 mM phenylmethylsulfonyl fluoride, and proteinase inhibitor cocktail). Detailed subsequent steps are described below. The Southern blots were performed based on manufacture's protocol of DIG high prime DNA labelling and detection starter kit II (Roche, Penzberg, Germany). The transplastomic plants were grown in hydroponic system for 2 weeks with AerogarGarden liquid plant food (Miracle-Gro, Marysville, Ohio) and then transferred to greenhouse for harvest. In the greenhouse, the soil was mixed with a 1:1 ratio of garden soil (Miracle-Gro, Marysville, Ohio) and potting soil (Erthgro), and the plants were fertilized with Miracle-Gro Water Soluble All Purpose Plant Food once or twice per week under the conditions of 16-h: 8-h light cycle at 22° C. Harvested leaves were lyophilized as previously described (Kwon et al., 2016).

### Quantitation and Characterization of CTB/PTD-Pro-IGF-1 in Lyophilized Cells

To confirm the CTB/PTD-Pro-IGF-1 expression levels, 10 mg of lyophilized leaf power was rehydrated in 500 μL of extraction buffer (100 mM NaCl, 10 mM EDTA, 200 m Tris-Cl, pH 8, 0.05% v/v Tween 20, 0.1% v/v SDS, 400 mM Sucrose, 14 mM β-mercaptoethanol, 100 mM DTT, 2 mM phenylmethylsulfonyl fluoride, and proteinase inhibitor cocktail) for 1 hour at 4° C. and was sonicated [three times for 5 seconds on/10 seconds off by Sonicator 3000 (Misonix, Farmingdale, N.Y.)]. After the extracted proteins were quantified by Bradford assay (Bio-rad Laboratories, Hercules, Calif.), they were heated at 70° C. in 1× Laemmli buffer for 10 min and separated on SDS-PAGE gels (12%). Western blot analysis was performed against rabbit anti-CTB (1:10,000) (Gen Way Biotech, San Diego, Calif.), rabbit anti-IGF-1 (1:4,000) (Abcam, Cambridge, UK), and goat anti rabbit IgG-HRP (1:4,000) (Southern Biotechnology, Birmingham, Ala.). The Precision plus protein standards were detected using Precision protein Strep Tactin conjugated and labelled with HRP (horseradish peroxidase) (1:5000) (Bio-rad Laboratories, Hercules, Calif.). Chemiluminescent based detection kit was used to capture the HRP signals on to X-ray films, and then quantification analysis was performed with ImageJ software (IJ 1.46; NIH). Standard curves were made based on CTB peptide (Sigma, St Louis, Mo.) or IGF-1 peptide (Abcam, Cambridge, UK).

### Immunoblots of CTB-Pro-IGF-1 in Non-Reducing Gels

Ten mg of lyophilized powder was rehydrated in 500 μL of extraction buffer (100 mM NaCl, 10 mM EDTA, 200 m Tris-Cl, pH 8, 0.05% v/v Tween 20, 0.2% v/v SDS, 400 mM Sucrose, 2 mM phenylmethylsulfonyl fluoride, and proteinase inhibitor cocktail) for 1 hour at 4° C., sonicated, quantified as described above. After the extracted proteins were mixed with sample buffer (250 mM Tri-Cl, pH 6.5, 10% w/v SDS, 50% v/v glycerol, 0.2 mM bromophenol blue), electrophoresis and western blot were performed. Detail subsequent steps of western blots were described above.

### Quantitation of Pro-IGF-1 in Fresh and Lyophilized Leaves

Equal amounts of fresh and lyophilized leaves were extracted in the same volume of extraction buffer, and then they were loaded in a serial dilution and run for immunoblots as described above. As for lettuce CTB-Pro-IGF-1 and marker-free PTD-Pro-IGF-1, 1× indicates 10 μl loading from 10 mg of lyophilized powder resuspended in 300 μL of protein extraction buffer. As for tobacco CTB-Pro-IGF-1, 1× indicates 1 μl loading from 10 mg of lyophilized powder resuspended in 300 μl of protein extraction buffer. For loading controls, the membrane was incubated in Western Stripping Buffer (Thermo Fisher Scientific, Waltham, Mass.) for 10 minutes at 37° C., then immunolabeled with rabbit anti-RbcL (Rubisco Large Subunit) (Agrisera, Vannas, Sweden) in PTM 1:40,000 for 1 hour. The subsequent western blot steps were followed as described above.

### GM1-Ganglioside Receptor Binding Assay

CTB-Pro-IGF-1 expressed in tobacco and lettuce were evaluated CTB pentamer form binding to GM1-ganglioside receptor after lyophilization. Proteins were extracted as described above. CTB-GM1 binding assay was carried out as described previously (Kwon et al., 2017).

Purification of CTB-Pro-IGF-1 and Cell Proliferation assay

Four hundred milligrams of lyophilized and ground tobacco transplastomic lines containing CTB-Pro-IGF-1 were rehydrated in 20 mL of plant protein extraction buffer (50 mM NaP (pH 8.0), 300 mM NaCl, 1.4 mM beta-mercaptoethanol, 0.5% v/v Tween 20, and 2 tablets of protease inhibitor cocktail) for 1 hour at 4° C. and homogenized. The homogenate was spun down after sonication. The supernatant was incubated with 2 mL of Ni resin (Clontech, Fremont, Calif.) for overnight. Once it was stacked up in columns, they were washed in a sequence with 10 mL of binding buffer (50 mM NaP (pH 8.0), 300 mM NaCl), 10 mL of wash buffer 1 (50 mM NaP (pH 7.0), 300 mM NaCl), and 10 mL of wash buffer 2 (50 mM NaP (pH 6.0), 300 mM NaCl). Then, they were eluted with elution buffer (50 mM (pH 6.0), 300 mM NaCl, 250 mM imidazole). Purified CTB-Pro-IGF-1 was evaluated on a Coomassie blue stained gel and its concentration was calculated based on standard curves made with known concentration of IGF-1 peptide (Abcam, Cambridge, UK) on the same protein gel followed by immunoblot analysis as described above. Certain number of cells [2,500 of HOK (Human Oral Keratinocytes), 5,000 of GMSCs (Human Gingiva derived Mesenchymal Stromal Cells), 3,000 of SCCs (Human head and neck Squamous Carcinoma Cells), and 4,000 of MC3TC (Mouse Osteoblast Cells) in 100 μL growth media] were seeded in three 96-well plates. After 16 hours at 37° C. with 5% CO₂, a series concentration of the purified CTB-Pro-IGF-1 and human IGF-1 commercial peptide (Abcam, Cambridge, UK) were incubated with the cells after diluted in 10 μL of PBS. Ten μL of PBS added in the cells was used as negative control for 0 ng of peptide. After 24, 48, and 72 hours, absorbance of viable proliferated cells were measured by MTT assay (Roche, Basel, Switzerland) at 570 mm with references at 655 nm. The data was biologically repeated twice run in triplets.

To examine osteogenic differentiation, MC3T3 cells were treated with osteogenic medium (OS medium). OS medium is α-MEM (Gibco) containing 10% FBS, 10 mM β-glycerophosphate (Sigma, St Louis, Mo.), 50 μg/ml ascorbic acid (Sigma) and 10⁻⁸ M dexamethasone (Sigma). After 5 days treatment with OS medium and purified CTB-Pro-IGF-1 at 100 ng/ml, 200 ng/ml and 300 ng/ml, cells were isolated for RNA extraction and qPCR analysis for osteogenetic markers: Alkaline phosphatase (ALP), runt related transcription factor 2 (RUNX2) and Osterix (Osx).

### Animal Studies

C57BL/6J mice 8 to 10 weeks of age were purchased from the Jackson Laboratories and housed in pathogen-free conditions at the University of Pennsylvania under institutionally approved protocols. Lyophilized plant cells were rehydrated in PBS to a final volume of 200 μL per gavage dose (containing 5 μg of CTB or PTD tagged IGF-1) and briefly vortexed for 3 to 4 seconds. Three groups of mice, 18 per group (9 males and 9 females) were fed using a 20 gauge gastric gavage needle. Blood (50-80 μL) was drawn from the submandibular vein before, and 2 hours after, gavage and allowed to clot at room temperature for 2 hours. Subsequently, sera were separated by centrifugation at 3000 RPM for 15 minutes at 4° C. and stored at −80° C. Levels of IGF-1 in sera were assessed by ELISA assay. Soleus or gastrocnemius muscles were dissected from the hind limb of controls and mice fed CTB or PTD tagged IGF-1, snap frozen in liquid nitrogen and assessed for IGF-1 by western blot.

### ELISA Assay of IGF-1 in Sera

IGF-1 standards (Abcam, Cambridge, UK) and sera samples were diluted in coating buffer (15 mM Na₂CO₃, 35 mM NaHCO₃, 3 mM NaH₃, pH9.6) and allowed to bind to Costar (3590) high-binding EIA/RIA plates overnight. Subsequently, plates were washed 3 times in PBS-0.1% Tween-20 (PBST), blocked with 3% milk in PBST (PTM) for 1 hour and incubated in primary antibody, rabbit anti-IGF-1 in PTM 1:2000 (Abcam, Cambridge, UK) for 2 hours at 37° C. Next, plates were washed 3 times in PBST and incubated in secondary antibody goat anti-rabbit IgG1 conjugated to HRP in PTM 1:4000 (Southern Biotechnology, Birmingham, Ala.) at 37° C. for 1 hour. Plates were then washed in PBST, incubated in TMB peroxidase substrate (Rockland) for 10 minutes at room temperature, followed by stop solution (2 N H₂SO₄). Absorbance was read at 450 nm.

### Immunoblotting Analysis of Muscle Tissues

Soleus and gastrocnemius muscle tissues were thawed from liquid nitrogen and homogenized in lysis buffer (10 volumes per muscle wet weight) consisting of 50 mM Tris-HCL, pH 7.4; 0.25% sodium deoxycholate; 150 mM NaCl and 1% Triton X-100; protease and phosphatase inhibitors (Sigma, St Louis, Mo.). Tissue homogenates were assessed for protein by Bradford assay (Bio-rad Laboratories, Hercules, Calif.). Tissues were analyzed by electrophoresis and western blotting as described above. For loading controls, the membrane was incubated in Western Stripping Buffer (Thermo Fisher Scientific, Waltham, Mass.) for 5 minutes at 37° C., was incubated in primary antibody mouse anti-actin (Santa Cruz Biotechnology, Dallas Tex.), 1:4000 or mouse anti-GAPDH (Abcam, Cambridge, UK), 1:4000 in PTM, washed in PBST, and incubated in HRP conjugated goat-anti-mouse IgG (Southern Biotechnology, Birmingham, Ala.) 1:4000 and developed by chemiluminescence.

### Murine Diabetic Femur Fracture Model

Twenty-five eight-week old C57BL/6J male mice from Jackson Laboratory were randomly assigned to four groups: CON group (6 male non-diabetic mice, oral gavage with lyophilized wide type (WT) plant cells), IGF-1 group (6 male non-diabetic mice, oral gavage with lyophilized plant cells containing CTB-Pro-IGF-1), Dia group (7 male diabetic mice, oral gavage with lyophilized WT plant cells), Dia-IGF-1 group (6 male diabetic mice, oral gavage with lyophilized plant cells containing CTB-Pro-IGF-1). WT plant cells powder and lyophilized plant cells (20 mg) containing 5 μg CTB-Pro-IGF-1 were respectively rehydrated in PBS to a final volume of 300 μl per gavage dose. The period of oral gavage was 6 weeks and the frequency of it was once a day.

Mice from Dia and Dia-IGF-1 groups were given daily intraperitoneal injections with STZ (50 μg/g body weight in 0.1M citrate buffer) for 5 days to induce diabetes at 3 weeks before fracture. Blood glucose levels were examined 7 days after the final streptozotocin injection by obtaining blood from tail vein and measuring glucose concentration with a glucometer (ONE TOUCH Ultra 2, LifeScan, Switzerland). Mice with blood glucose levels greater than 300 mg/dl were considered diabetic.

Closed femoral fractures with intramedullary nail fixation were performed in 12-week (wk)-old mice described above. Briefly, closed fractures were generated by a three-point blunt guillotine driven by a dropped weight, which creates a uniform transverse fracture of the femur. The fractured femurs were harvested at 6 weeks post fracture for analysis.

Micro-Computed Tomography (microCT) Imaging and Radiography

Mice were anesthetized by isoflurane and the fracture sites were radiographed using a Faxitron MX-20 (Faxitron X-Ray) at D0, D7, D14 and D21 to monitor the progress of fracture healing. At the end of this study, bone microarchitecture of the fractured femur was tested using Scanco Medical CT 35 at the Imaging Core, Penn Center for Musculoskeletal Disorders. Scans were performed at 55 keV and 3D images were reconstructed at the fracture line extending at least 3 mm to the proximal and distal. The ratio of the bone volume to the total volume (BV/TV) and bone density were determined in the fracture callus with density thresholds set at 300 mg/cm³ HA.

### Histological Analyses

Fracture calluses were excised, fixed with 4% paraformaldehyde and decalcified in 10% ethylenediaminetetraacetic acid (EDTA) for a minimum three weeks at 4° C. The samples were then embedded in OCT. Longitudinal sections (8 μm thick) were cut from the mid-portion of the callus and mounted serially on gelatin-coated glass slides and stained with hematoxylin and eosin (Sigma-Aldrich) for histological examination. The morphological characteristics of bonehealing were observed by Leica fluorescent microscope (DMI6000B, Leica, Germany).

The following examples are provided to illustrate certain embodiments of the invention. They are not intended to limit the invention in any way.

### Example I

### Generation of Antibiotic Free IGF-1 in Chloroplasts

In this study, we generated lettuce and tobacco lines expressing codon optimized Pro-IGF-1 containing e-peptide fused with transmucosal carriers Cholera non-toxic B subunit (CTB) or cell penetrating protein transduction domain (PTD) in chloroplasts. We achieved complete deletion of the antibiotic resistance gene (aadA) and observed stable maintenance and expression of Pro-IGF-1 in the next generations. Biological activities of chloroplast derived Pro-IGF-1 were confirmed in vitro by observing enhanced proliferation of four different human/mouse oral cells. Utilizing both male and female mice, we observed that IGF-1 produced in transplastomic plants can be successfully delivered orally to the circulation and bone tissues. Administration of IGF-1 by oral gavage significantly promoted osteoblast differentiation and diabetic fracture healing, confirming potential for translation of this unique biopharmaceutical to human studies. Together with the complete removal of the antibiotic resistance gene, this novel platform can be used in clinical studies of bioencapsulated biopharmaceuticals for affordable oral drug delivery and enhanced patient compliance.

### Results

### Generation of Lettuce/Tobacco Transplastomic Lines Expressing CTB/PTD-Pro-IGF-1

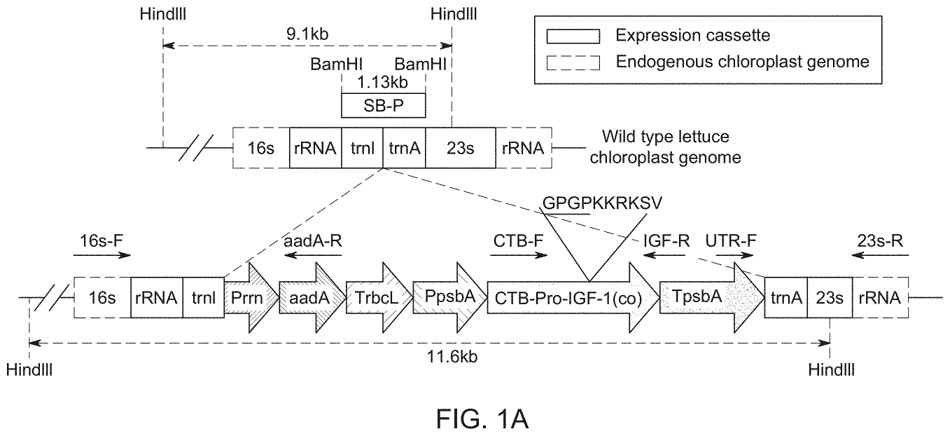

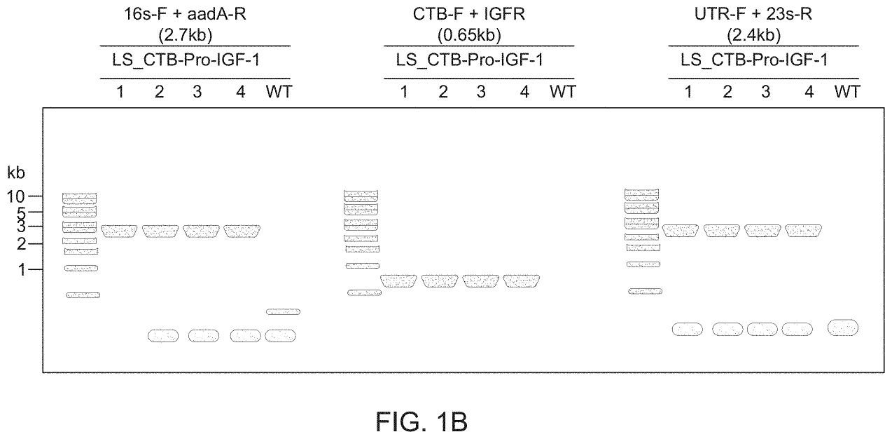

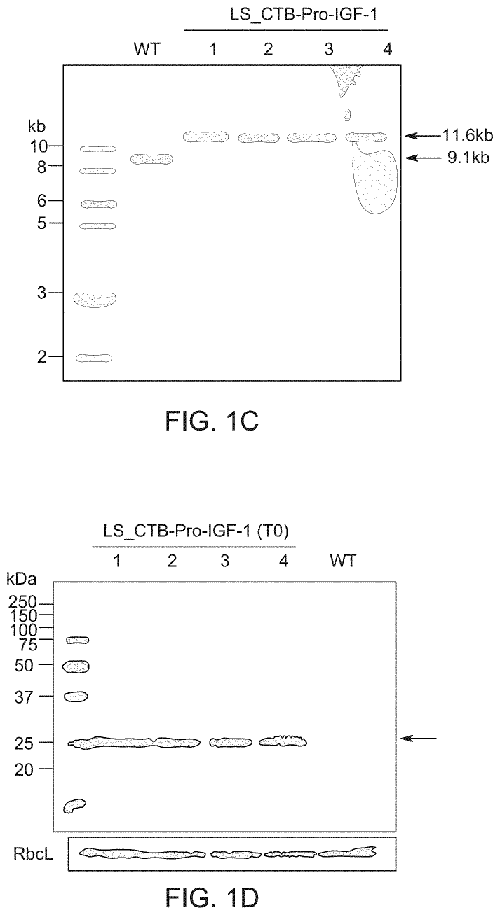

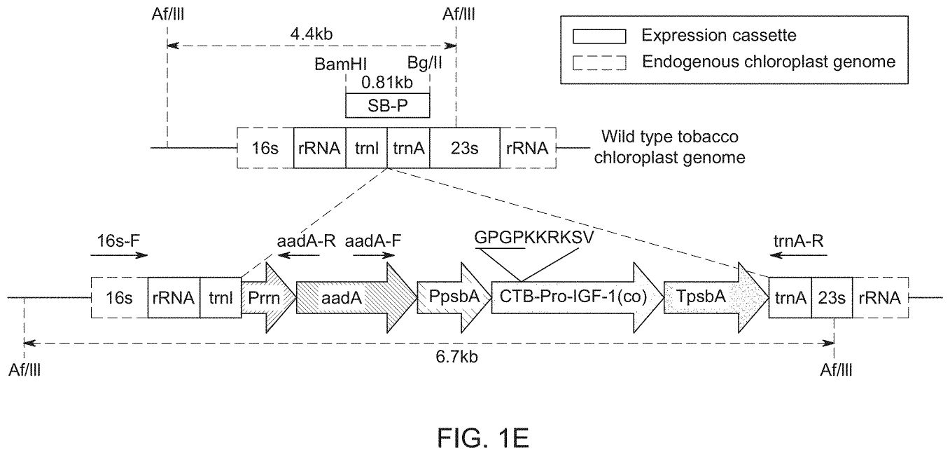

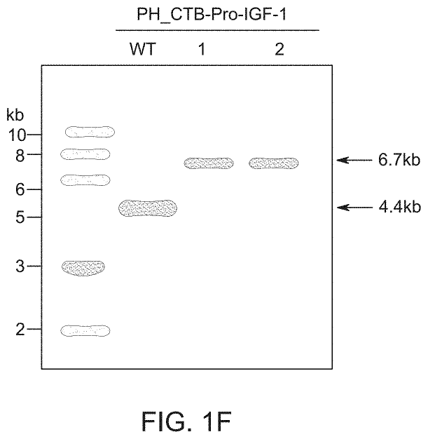

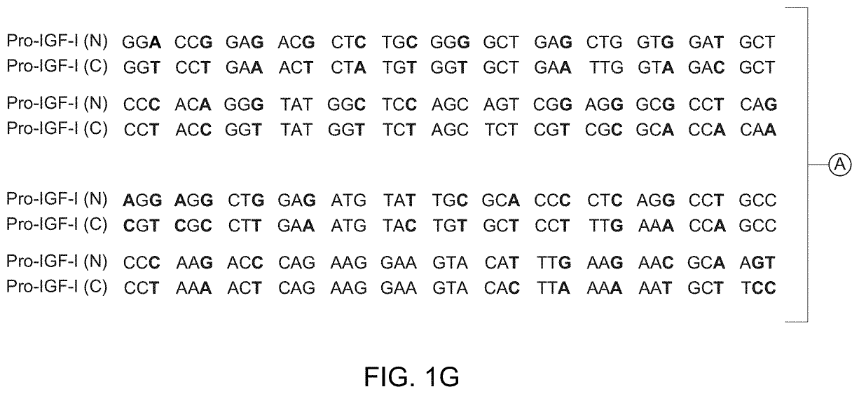

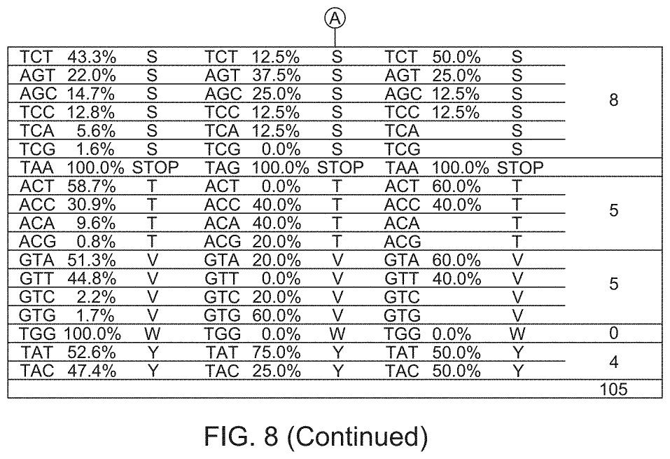

A precursor form of human IGF-1 with e-peptide (Pro-IGF-1) was synthesized after codon optimization to improve its expression level in chloroplasts, using the psbA gene as reference (Kwon et al., 2016). Out of total 105 codons of Pro-IGF-1, 65 codons were optimized resulting in increased AT content from 43% to 57%. In addition, three amino acids, Lsy68, Arg74 and Arg77, were changed to Gly68, Ala74 and Ala77, respectively, to avoid the recognition by endoprotease so as to prevent e-peptide cleavage (Duguay et al., 1995) (FIG. 1G and FIG. 8). Predicted glycosylated sites were not edited in this study because it is well-known that chloroplasts are a glycosylation free zone and offer protection from glycosylation even with potential glycosylated sites.

The codon-optimized Pro-IGF-1 was fused to CTB via a hinge (GPGP) and a furin sequence (KKRKSV), and the fusion construct was cloned into lettuce (pLsLF) (FIG. 1A) and tobacco (pLD-utr) chloroplast transformation vectors (FIG. 1E). The plasmids were bombarded into lettuce/tobacco chloroplast genomes as described previously (Kwon et al., 2017; Verma et al., 2008). After the bombardment, regenerated shoots in the first round of selection were tested using PCR analysis with specific primer sets (FIG. 1B and FIG. 1E) to confirm site specific integration of the expression cassettes (FIG. 1B). PCR-positive shoots were subject to another round of selection to achieve homoplasmy. The homoplasmic states were confirmed by Southern blots assay using suitable probes (SB-P) (FIG. 1C and FIG. 1F) and the expression of the CTB-Pro-IGF-1 was detected in western blots (FIG. 1D). The transformed chloroplast genomes showed distinct fragments of 11.6 kb in lettuce and 6.7 kb in tobacco confirming site-specific integration of CTB-Pro-IGF-1 expression cassettes compared to smaller fragments in the untransformed chloroplast genomes (9.1 kb in lettuce and 4.4 kb in tobacco). The western blot showed expression of CTB-Pro-IGF-1 protein of the expected size (24.3 kDa). These results confirmed the generation of lettuce/tobacco homoplasmic lines expressing CTB-Pro-IGF-1.

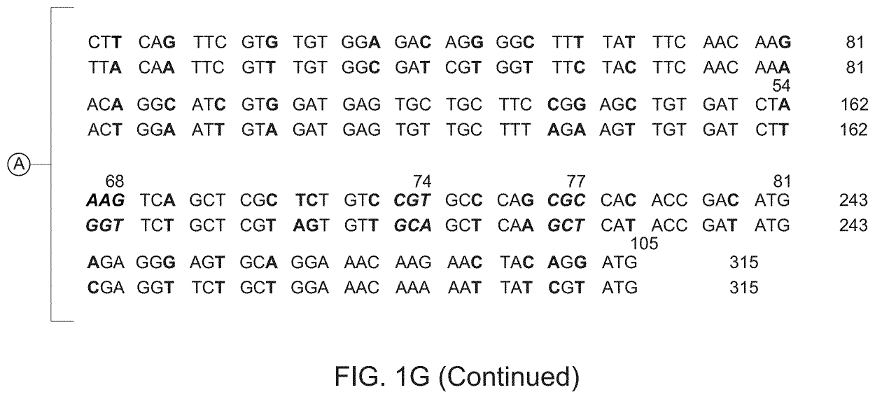

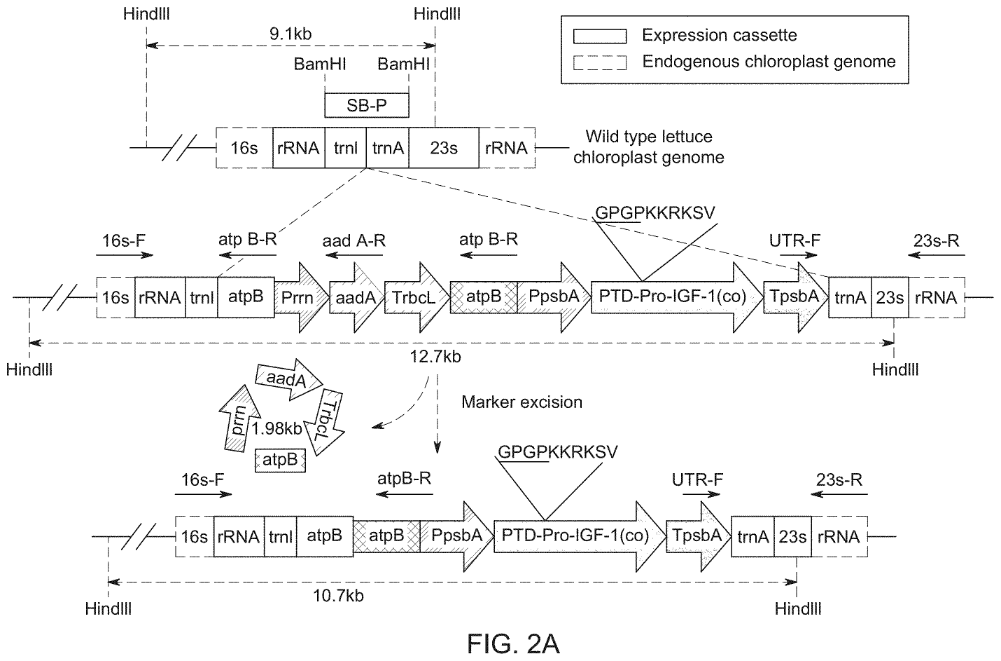

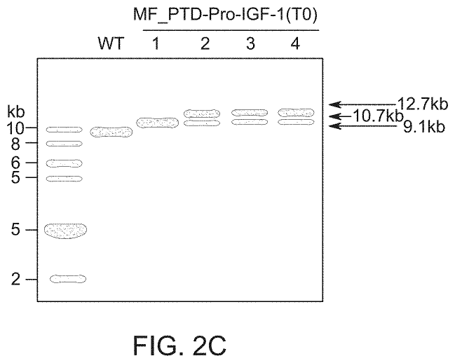

Generation of Marker-Free PTD-Pro-IGF-1 Homoplasmic Lettuce Lines The marker-free chloroplast vector (pLsLF-MF) was generated by multiple cloning steps (FIG. 2J) containing the spectinomycin resistant aminoglycoside-3′-adenylyl-transferase (aadA) gene under the control of plastid ribosomal RNA promoter (Prrn) and followed by 3′UTR, TrbcL. The aadA is located between two copies of chloroplast encoded CF1 ATP synthase subunit beta (atpB) promoter region (649 bp) as shown in FIG. 2A. The codon-optimized synthetic Pro-IGF-1 fused to PTD was cloned into the marker-free chloroplast expression cassette, under the control of the psbA promoter/5′ UTR and 3′ UTR. The bombarded lettuce leaves were regenerated on spectinomycin (an inhibitor of plastid protein synthesis which binds the 30S ribosome) containing media and subsequently screened with specific sets of PCR primers during the first (data not shown) and second round of selections (FIG. 2B). The 16s-F/aadA-R primer set anneals to the endogenous chloroplast genome sequence and the aadA transgene within the cassette (FIG. 2A), amplifying a 3.3 kb DNA fragment (FIG. 2B). The set of UTR-F/23s-R primers was used to verify the 3′ side of the expression cassette (FIG. 2A), and produced a fragment of 2.4 kb (FIG. 2B).

During the selection of lettuce chloroplast transformants on spectinomycin, the selection marker was excised, leaving one copy of the atpB region in the transplastomic genome by homologous recombination between the two directly repeated atpB fragments. Once the homology-based marker excision happens, the primer set of 16s-F/atpB-R should amplify a 2.4 kb PCR product and the 16s-F/aadA-R should not produce any PCR products. As shown in FIG. 2B, transplastomic line 1 showed no PCR product when amplified with the 16s-F/aadA-R primer set, resulting from the excision of the aadA gene, and the presence of an amplified 2.4 kb fragment with the 16s-F/atpB-R. This excision didn't affect the stable integration of the PTD-IGF-1 expression cassette, which was confirmed by the PCR amplification of a 2.4 kb fragment using the UTR-F/23s-R primer set. In lines 2, 3, and 4, 3.3 kb PCR products were produced by the 16s-F/aadA-R, indicating that aadA marker genes were still present in the chloroplast genomes (FIG. 2B). Three primary shoots showed positive results in the 1st PCR screening; among 9 shoots screened in the 2nd round selection from one of the 3 primary, 6 shoots were positive for transgene cassette integrations and one shoot showed complete removal of the aadA gene.

The PCR positive shoots (line 2, 3 and 4) in FIG. 2B were examined along with the antibiotic resistance gene-free shoot (line 1) and untransformed wild type lettuce by Southern blot analysis (FIG. 2C). In the Southern blot, while untransformed lettuce showed a single 9.1 kb fragment, transplastomic line 1 showed a 10.7 kb fragment supporting the PCR results (FIG. 2B) and achievement of homoplasmic marker-free line (marker gene excision from all copies of the chloroplast genome). Interestingly, in line 2, 3, and 4, two fragments at 12.7 kb and 10.7 kb in size were detected, which represents a heteroplasmic state with or without the marker excision. Regardless of aadA gene excision, the expression of PTD-Pro-IGF-1 proteins was detected at the expected size, 14.5 kDa, in all the examined lines when using IGF-1 antibody, while no band was detected in the untransformed wild type lettuce (FIG. 2D).

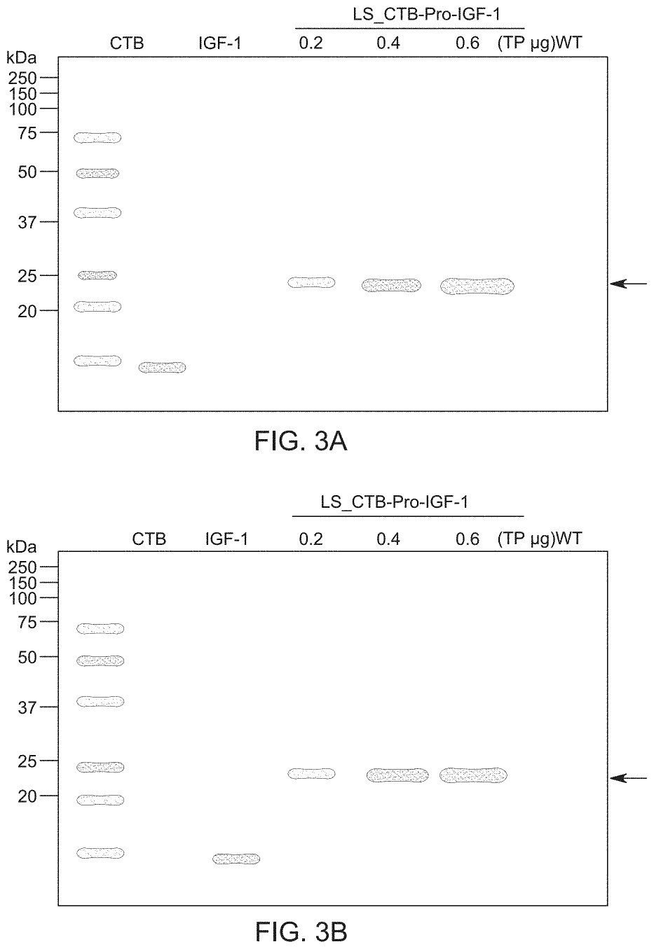

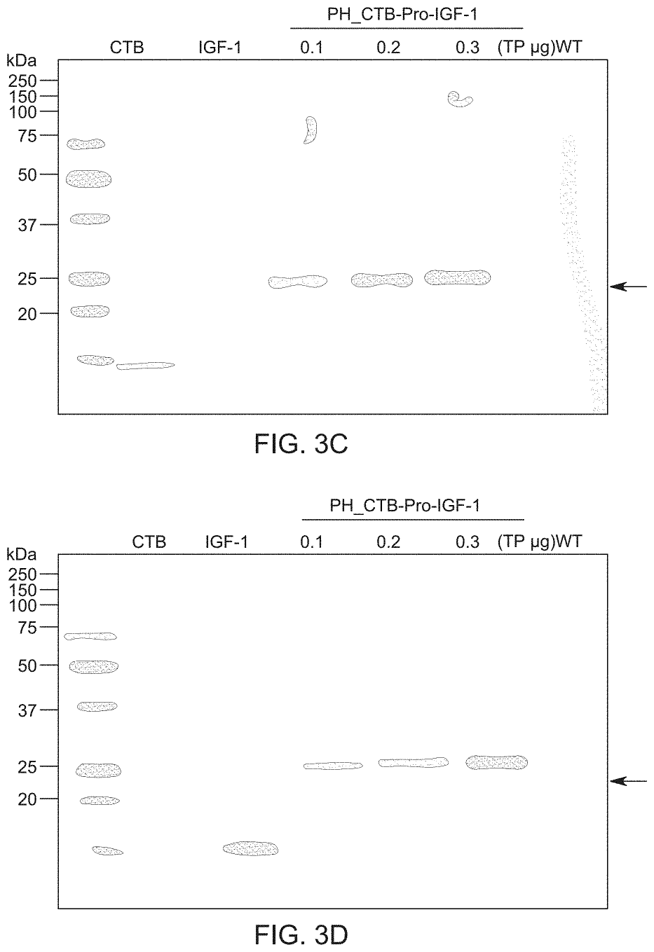

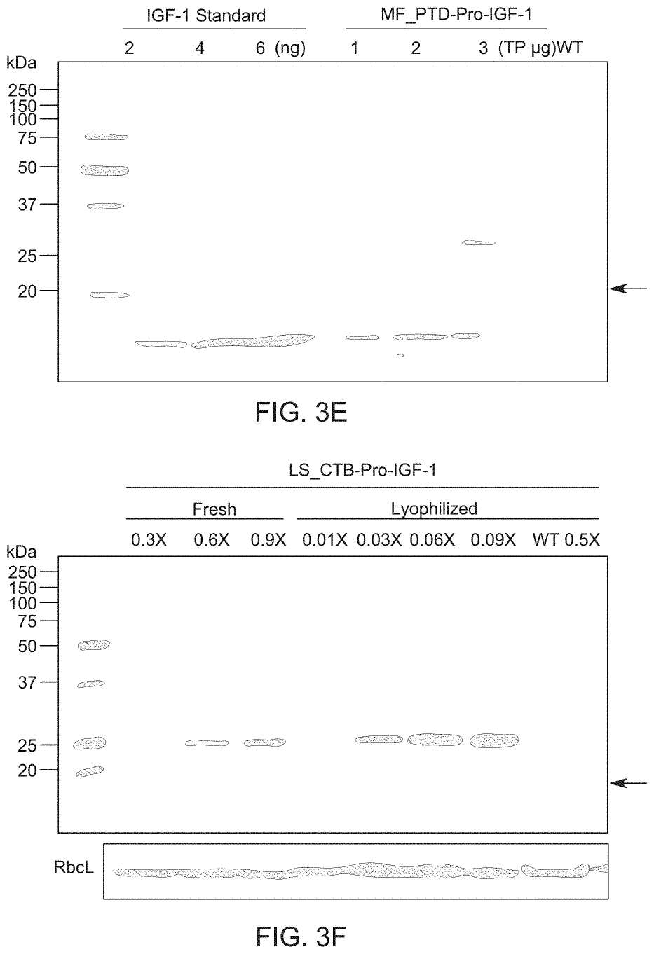

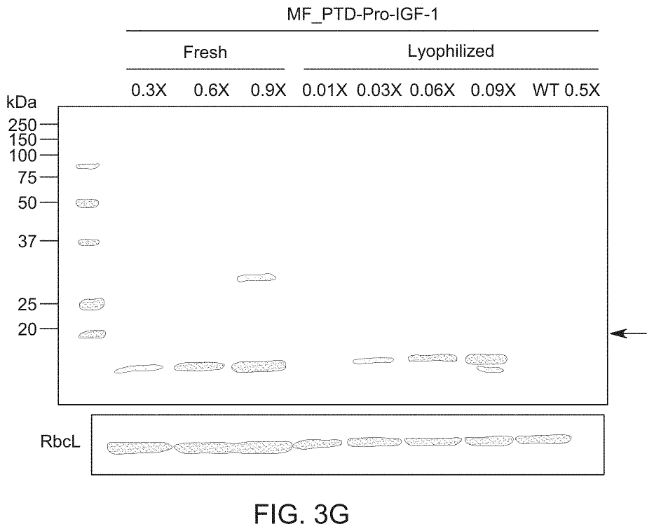

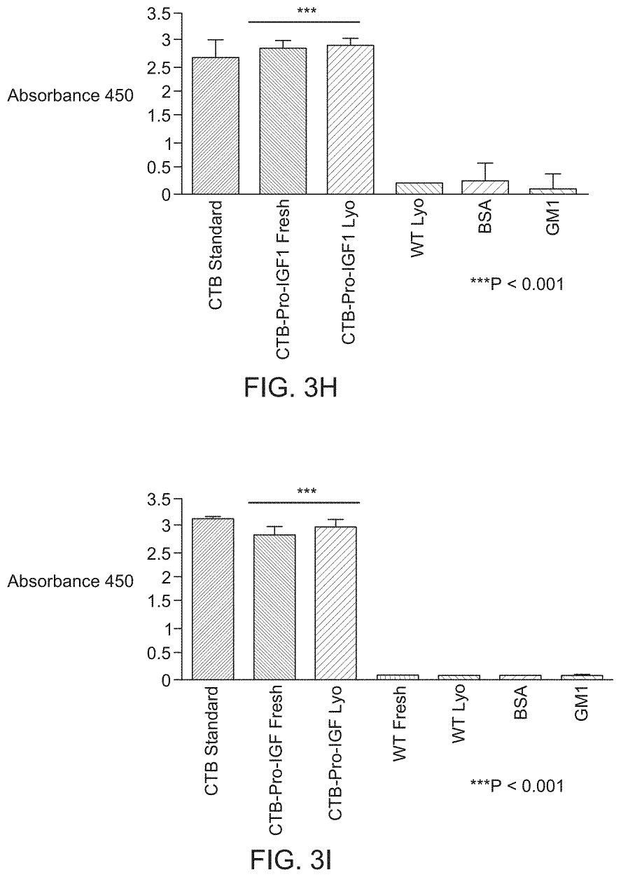

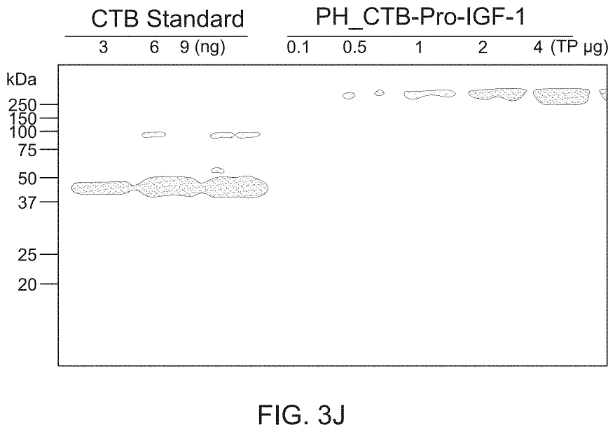

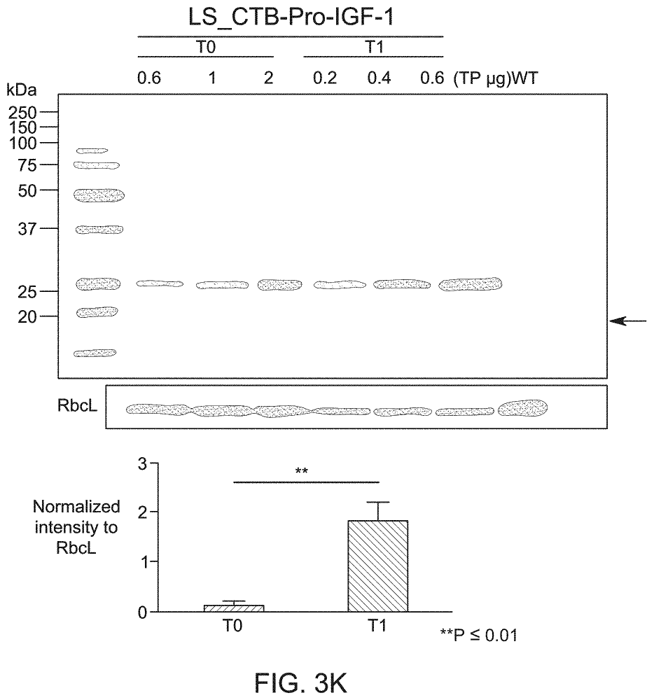

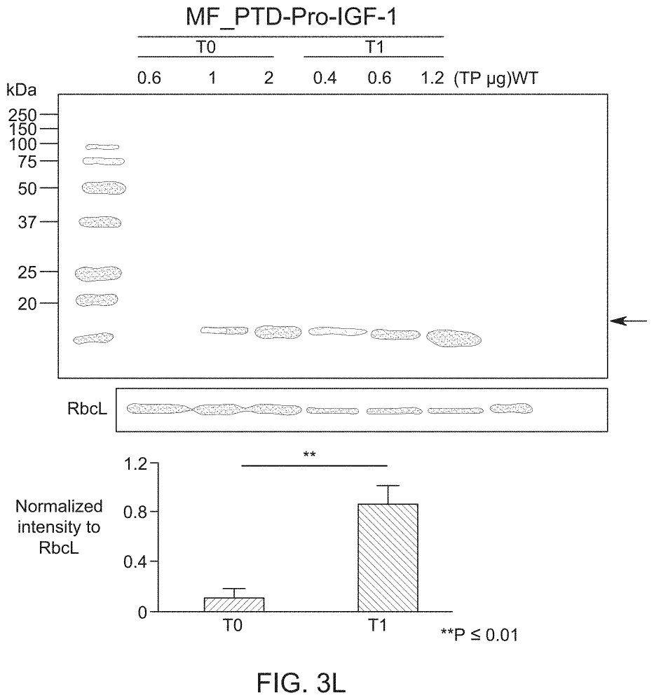

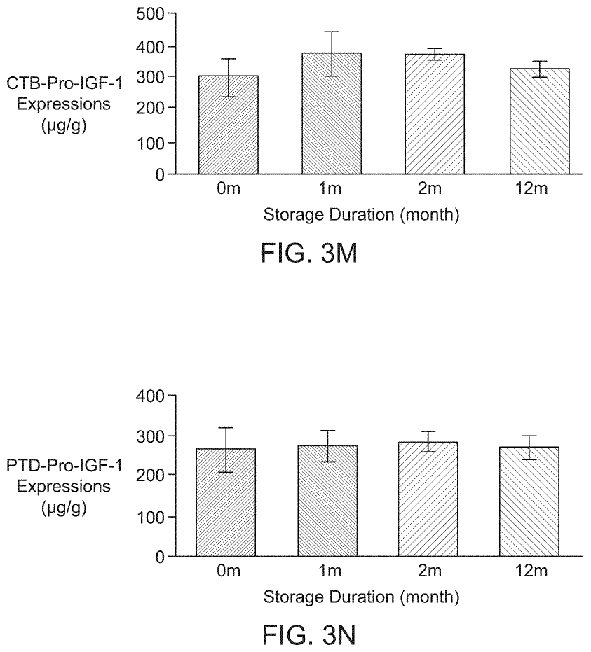

To evaluate stability of the marker-free homoplasmy in the next generation, 10 marker-free seeds were germinated on spectinomycin-free media. Genomic DNA was extracted from the T1 plants and examined by PCR with the same primer sets used in T0 transplastomic line evaluation and Southern blots were carried out (FIG. 2E and FIG. 2F). All the examined seedlings showed antibiotic gene excision but maintained the integration of the PTD-Pro-IGF-1 expression cassette. Expression of PTD-Pro-IGF-1 in the T1 generation was also confirmed by a western blot against anti-IGF-1 (FIG. 2G). As shown in the T0 plants (FIG. 2D), all examined T1 plants expressed PTD-Pro-IGF-1 after marker gene excision. Also, when T1 antibiotic-free transplastomic lines were germinated on antibiotic containing media (spectinomycin 25 and 50 μg/mL), the leaves bleached out in 6 days after germination and the plants stopped growing 15 days after germination (FIG. 3O). Four randomly selected lines of T2 marker-free plants also showed the antibiotic resistance gene-free homoplastic state expressing Pro-IGF-1, as confirmed by Southern blot and immunoblot analysis, respectively (FIG. 2H and FIG. 2I). These data confirm that the marker-free state is stably maintained through subsequent T1 and T2 generations, while maintaining expression of the foreign protein, Pro-IGF-1.

### Characterization of CTB/PTD-Pro-IGF-1 Expression in Lyophilized Plant Cells

Expression of CTB-Pro-IGF-1 in both lettuce (FIGS. 3A and 3B) and tobacco (FIGS. 3C and 3D) chloroplasts was evaluated using antibodies against CTB or IGF-1 and the two different antibodies detected same size target protein. Based on the standard curve generated using known amounts of IGF-1 peptide, the concentration of expressed PTD-Pro-IGF-1 in lyophilized lettuce leaves was extrapolated 270 μg/g dry weight (FIG. 3E). Likewise, when using a CTB standard curve, the expression level of CTB-Pro-IGF-1 was estimated 370 μg/g in lyophilized lettuce T0 lines (data not shown). The concentration of the expressed therapeutic protein was compared between fresh and lyophilized cells using immunoblot analysis with antibody against CTB or IGF-1 (FIGS. 3F and 3G). Expression level of both CTB- and PTD-Pro-IGF-1 was ˜10-fold higher in the lyophilized samples when compared to those in fresh leaves based on weight.

The pentameric form of CTB, which is created by disulfide bonds, 30 hydrogen bonds, 7 salt bridges and several hydrophobic interactions among CTB monomers, can bind to GM1 receptor (Sanchez et al., 2008). CTB carries any fused proteins across gut epithelial cells by binding to GM1 receptors so that the fused protein can be delivered to the circulatory system after the cleavage from CTB by ubiquitous protease furin (Kwon et al., 2016). To investigate formation of the pentameric CTB structure, GM1 binding ELISA was performed using fresh and lyophilized leaf materials for each lettuce (FIG. 3H) and tobacco (FIG. 3I) plant cell expressing CTB-Pro-IGF-1. The absorbance values of the fresh and lyophilized cells expressing the CTB fusion proteins at 450 nm were as high as CTB standard proteins (positive control) with no significant signals in negative controls. These results show that the expressed CTB-Pro-IGF-1 fusion proteins in chloroplasts can be properly folded in the pentameric form and the integrity of the pentameric structure can be also maintained after lyophilization. The presence of the pentameric CTB-Pro-IGF-1 structures in the tobacco lyophilized materials was also confirmed using non-reducing SDS-PAGE (FIG. 3J). The size of the pentameric form of CTB-Pro-IGF-1 is approximately 121.5 kDa but a larger size was observed, which is probably a dimer form of the pentameric structure. This may be due to the strong interactions between CTB pentamers in the absence of reducing agents (Sanchez et al., 2008) and/or inter or intra molecular disulfide bonds in CTB-IGF-1. Most importantly, the single protein band in FIG. 3J supports existence of only the assembled CTB-Pro-IGF-1 pentamers in plant cells and absence of any cleaved or monomeric products with remarkable stability.

Expression levels of Pro-IGF-1 in the transplastomic lines were significantly increased in next generation (FIGS. 3K and 3L). The level of both CTB- and PTD-Pro-IGF-1 was higher in T1 lines than that of T0 lines with an average of 15-fold in CTB- and 7-fold in PTD-Pro-IGF-1, irrespective of the marker removal or the kind of fusion tag. Both CTB- and PTD-Pro-IGF-1 fusion proteins in lyophilized lettuce materials kept stable up to 12 months at an ambient temperature (FIGS. 3M and 3N). No significant change was found in the level of IGF-1 expressed in the lyophilized powder (IGF-1 μg/g Dry Weight) during long-term storage, with an average of 370 μg/g of CTB- and 270 μg/g of PTD-Pro-IGF-1, when the same batch of plant powder was examined in western blots at different storage time points, regardless of the selectable marker excision.

### Cell Proliferation Induced by Chloroplast Expressed CTB-Pro-IGF-1

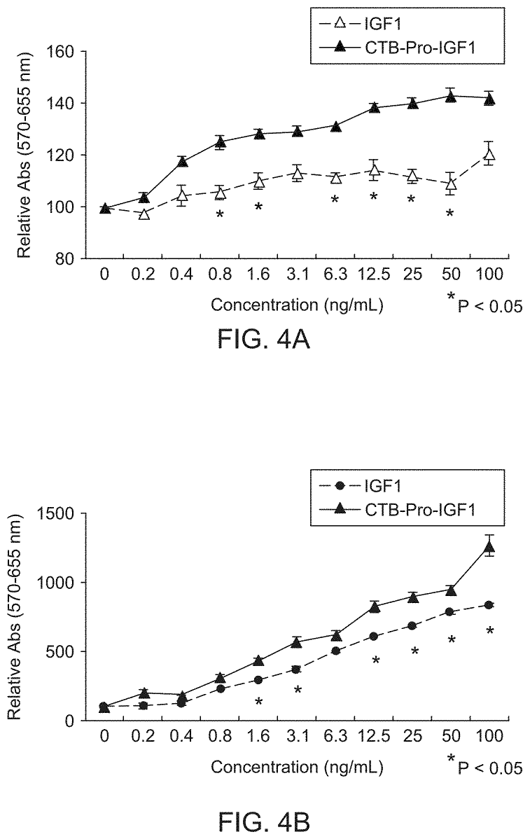

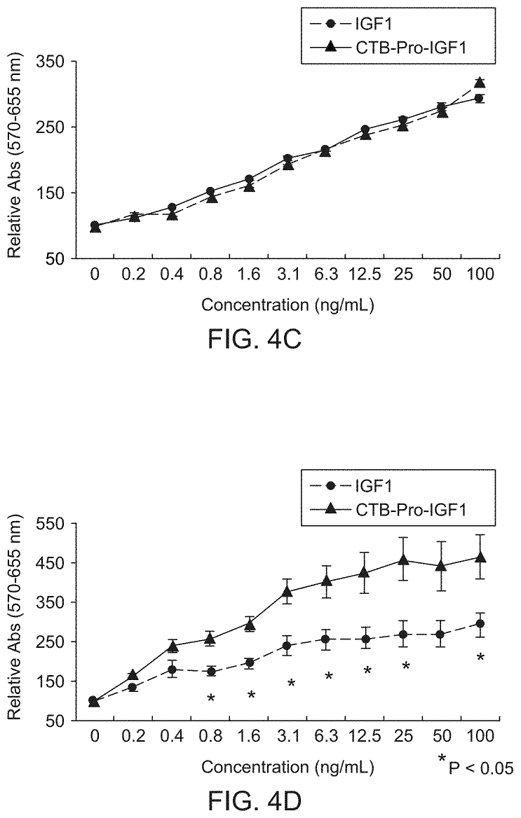

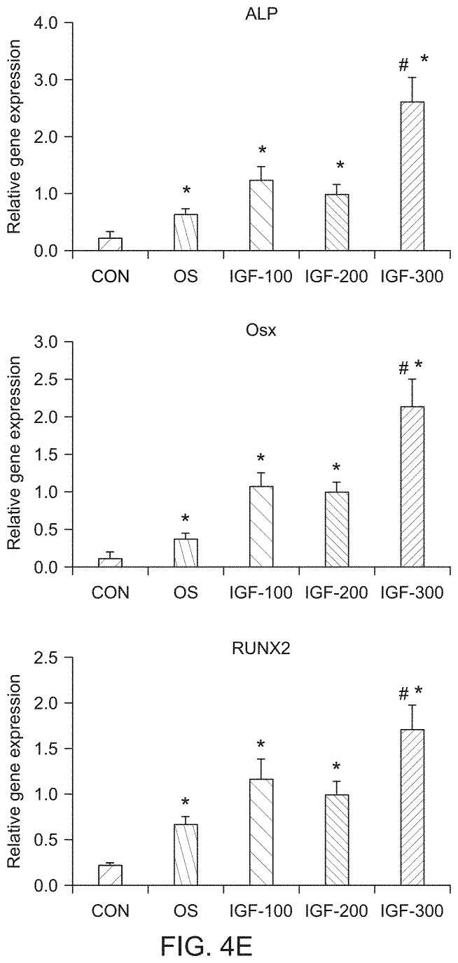

To evaluate cell proliferation effects of the plant derived CTB-Pro-IGF-1 in vitro, the MTT assay was performed on four different human and mouse cell types: HOK (Human Oral Keratinocytes), GMSCs (Human Gingiva-derived Mesenchymal Stromal Cells), SCCs (Human head and neck Squamous Carcinoma Cells), and mouse osteoblasts (MC3T3). CTB-Pro-IGF-1 was purified from lyophilized plant cells using the affinity of nickel binding to the cluster of imidazole rings created by the resultant proximity of histidine residues when CTB monomers come close and form the pentameric CTB-Pro-IGF-1 structures. After the purified chloroplast derived protein was evaluated by immunoblot analysis with human IGF-1 peptide as a control (data not shown), a serial dilution of the CTB-Pro-IGF-1 was incubated with for different oral cell types. Chloroplast derived CTB-Pro-IGF-1 stimulated proliferation of HOK (˜1.2-fold), GMSCs (˜1.7-fold) and mouse osteoblasts (˜1.7-fold) when compared to commercial IGF-1 (FIGS. 4A, 4B, and 4D) while similar effects on proliferation was observed in SCCs (FIG. 4C). These results demonstrate that the plant derived Pro-IGF-1 promotes proliferation in all four oral cell types in a dose dependent manner and the rate is higher on HOK, GMSCs, and mouse osteoblasts when compared to mature IGF-1, indicating that the Pro-IGF-1 expressed in plant chloroplasts is fully functional and more potent than the commercial IGF-1 product. To determine whether the IGF-1 can promote osteoblast differentiation, MC3T3 cells were treated with OS medium with or without purified CTB-Pro-IGF-1 for 5 days and then examined the gene expression of the osteogenesis markers (ALP, OSX, RUNX2). As expected, our data showed that treatment with CTB-Pro-IGF-1 at 300 ng/ml significantly up-regulated the expression of ALP, OSX, and RUNX2, demonstrating that IGF-1 can promote OB differentiation (FIG. 4E).

### Evaluation of Oral Delivery of IGF-1

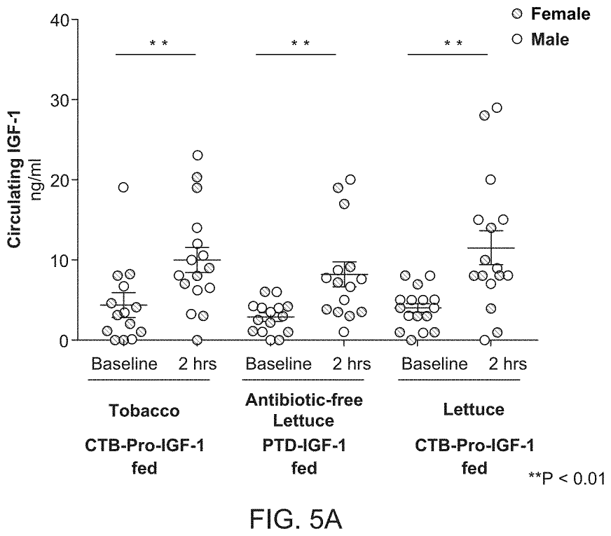

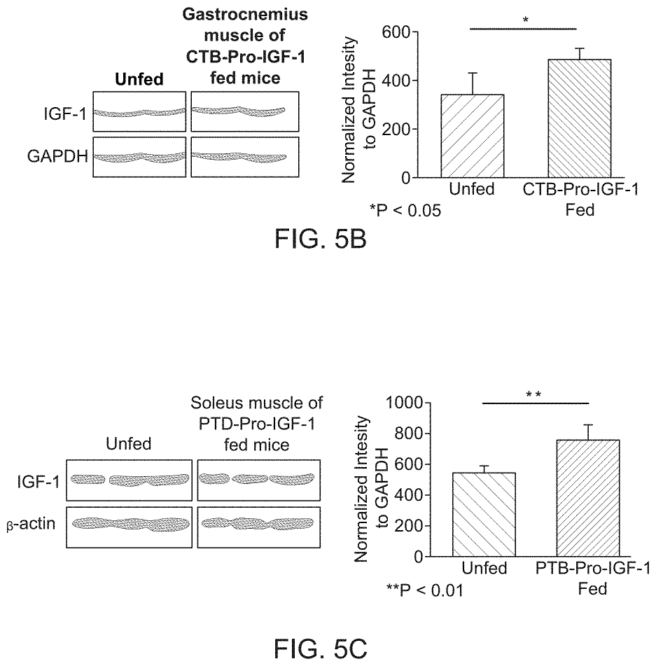

Lyophilized tobacco and lettuce plant cells expressing CTB-Pro-IGF-1 and PTD-Pro-IGF-1 or CTB-Pro-IGF-1, respectively, were suspended in PBS and administered to C57BL/6J mice by oral gavage. The level of IGF-1 was significantly increased in the circulation at 2 hours following feeding (FIG. 5A). Increases ranged from 2 to 3-fold (67% to 200%), with an average of 100% in IGF-1 observed from PTD-Pro-IGF-1 derived from marker-free lettuce and CTBPro-IGF-1 derived from either lettuce or tobacco. No difference in uptake between males and females was observed. These findings indicate that the pentameric CTB fusion proteins bound to GM1 receptors passed through the epithelia barrier via receptor-mediated trans-epithelial transport pathway. Also, the positively charged PTD domain can carry the fused proteins into cells by a receptor-independent fluid-phase micropinocytosis. Both transport systems were effective for the delivery of IGF-1 to the blood. Furthermore, the presence of increased IGF-1 in gastroenemius (FIG. 5B) and soleus muscle (FIG. 5C) at 5 hours after the oral gavage indicates that CTB and PTD act as transmucosal carriers for the delivery of IGF-1 to muscle tissues. IGF-1 was increased in both gastrocnemius and soleus muscle by approximately 28% at five hours after feeding (FIGS. 5B and 5C).

### Systemic Administration of IGF-1 Promotes Bone Regeneration in Diabetic Fracture Healing

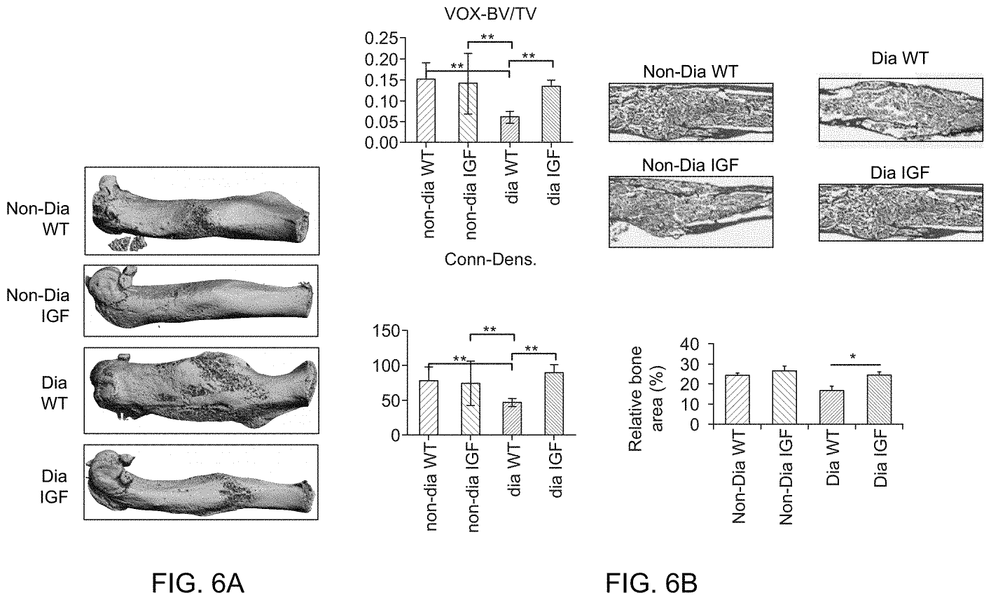

To further characterize the effect of CTB-Pro-IGF-1 on bone regeneration in vivo, we created a fracture model in both Diabetes (Dia) age and gender matched wild type (CON) mice. Micro-CT images, which covered around 3 mm proximal and 3 mm distal to the fracture site, 0 were acquired at 6 weeks post-fracture. As expected, 3D reconstruction of microCT images showed a significant reduction of bone formation in Dia mice compared to CON mice. Dia mice had much less bone mass with low-bone density and porous woven bone in the fracture sites compared to CON mice (FIG. 6A). Accordingly, the ratio of BV/TV and bone density were decreased by 54% and 39% respectively. Moreover, the histological analysis results showed that the relative bone area was reduced by 31% in Dia mice compared to the CON mice (FIG. 6B). The bone volume, density and area were significantly increased after IGF-1 treatment when compared to the WT plant treated Dia mice (FIG. 6A and FIG. 6B).

### Discussion

Production of biopharmaceuticals in plant cells was approved by the FDA in May 2012 (Xu et al., 2014), achieving one of the major goals of using plants for molecular farming applications. Unfortunately, glucocerebrosidase produced in plant cells (Devescovi et al., 2008) failed to provide anticipated cost effective benefit to Gaucher's disease patients when compared to its competitors such as Imiglucerase (produced by CHO cells) and velaglucerase (produced by human fibrosarcoma cells) (Taliglucerase for Gaucher Disease, 2012) due to high cost of purification, cold chain and injectable delivery. Attempts to reduce the cost through oral delivery of glucocerebrosidase was not successful (Shaaltiel et al., 2015), due to lower levels of expression. A landmark clinical trial showed that peanut allergy can be suppressed upon early introduction in small quantities and FDA approval is anticipated very soon (Tilles et al., 2018). This oral delivery approach has opened the door for using plant cell antigens and the gut immune system to treat a number of food or pollen allergies and suppress antibodies to injected protein drugs or deliver functional proteins.

Similarly, the plant chloroplast system has been advanced to deliver therapeutic proteins with various advantages including a high-level expression and ease of oral administration. However, the retention of the antibiotic resistance gene in transplastomic lines could pose hurdles in the regulatory approval process. Previous studies have shown that direct DNA repeats >600 bp promote homology-based marker excision in tobacco chloroplast genomes (Iamtham et al., 2000). More recently, we have demonstrated the first application of marker-free transplastomic lettuce expressing food/feed enzymes (Daniell et al., 2019 and Kumari et al., 2019). Therefore, to create the Pro-IGF-1, we used 649 bp of two atpB promoter regions to initiate the marker excision from the lettuce chloroplast genome. Interestingly, the aadA gene was successfully excised even in the first round of selection. This may be due to the copy correction mechanism that maintains identical inverted repeat regions (including spacer regions) found in >800 sequenced chloroplast genome (Daniell et al., 2016). When the aadA gene is not excised in early selection process in lettuce, its removal in the next generation by germinating seeds in antibiotic containing media is quite challenging. These results suggest that generation of maker-free transplastomic edible plants is efficient and precisely removes the selectable marker genes in a short, simple method with no need for additional transformation steps. Further, the excision of antibiotic resistance genes not only reduces metabolic load of the transplastomic crops but also enables the same selection marker to be reused for subsequent transformation of additional genes.

Previous studies suggested potential biological roles of the e-peptide. Pro-IGF-1 and Gly-Pro-IGF-1 are predominant rather than mature IGF-1 in skeletal muscle tissues and this proportion was maintained even after viral over-expression of the Igf1 gene. The e-peptide requirement for muscle mass was proposed based on lack of improvement on muscle hypertrophy in young mice after over-expression of IGF-1 and increased phosphorylation of cascade proteins was shown when Pro-IGF-1 was virally over-expressed (Philippou et al., 2014). Moreover, independent e-peptide has stimulated IGF-1R signaling and regulated cell proliferation and differentiation in various human cells and cell lines (Philippou et al., 2014). Hence, the retention of e-peptide expressing Pro-IGF-1 in the transplastomic lines generated in this study should increase the efficacy of IGF-1 treatment.

IGF-1 has been used as a therapeutic candidate to treat multiple muscle disorders. Circulating IGF-1 produced mostly in the liver is not adequate to improve muscle hypertrophy, therefore delivery to muscle tissue is required for efficacy. Boosting IGF-1 levels in the circulatory system is a suitable strategy since elevated IGF-1 levels in sera resulted in muscle mass enhancement due to their trans-localization to the muscle tissues (Philippou et al., 2014). It is well established that oral administration of untagged IGF-1 results in little or no-detectable IGF-1 in sera therefore IGF-1 has been administered by injections in clinical studies. No detectable IGF-1 was found in sera of pigs fed 3.4 mg/kg IGF-1 per day for four days (Burrin et al., 1996). Recombinant IGF-1 was increased in serum levels in humans approximately 100% over 20 days of 12 hourly injections in malnourished continuous ambulatory peritoneal dialysis (CAPD) patients (Fouque et al., 2000). In contrast, the present study demonstrates that orally delivered Pro-IGF-1 raised circulating levels of IGF-1 ˜100% at 2 hours and ˜28% increases in local muscle tissues at 5 hours after one gavage feeding in mice. Oral administration of IGF-1 is preferable to daily injections, which are necessary in patients due to the 12-hour half-life of IGF-1 in blood. These findings indicate that chloroplast derived Pro-IGF-1 can be an effective approach to treat muscle disorders by maintaining its functional characteristics and ease of oral delivery by absorption through intestinal epithelial cells and delivery into muscle tissue.

The IGF-1 signaling pathway is initiated by IGF-1 receptor (IGF-1R) phosphorylation upon IGF-1 binding. It involves the PI3K/Akt and MEK/Erk (Mitogen-activated protein kinase, MAPK) signaling pathways which were shown to enhance the pro-proliferative effects of IGF-1 in satellite cells and myoblasts, which results in muscle growth (Bikle et al.). In our study, no difference was detected between proliferation of squamous carcinoma cells exposed to commercial IGF-1 and CTB-Pro-IGF-1, probably due to extremely large number of IGF-1R (Rios et al., 2015), thereby effectively competing with CTB-GM1 binding on the cell surface. An alternative pathway by which IGF-1 interaction with its receptor induces cellular proliferation involves receptor-mediated endocytosis. Current evidence suggests that IGF-1 binding to the IGF-1R followed by IGF-1R internalization regulates the SHc/MAPK pathway. In the present study, we observed increased proliferation of Human Oral Keratinocytes (HOK), Human Gingiva-derived Mesenchymal Stroma Cells (GMSCs) and mouse osteoblasts (MC3T3) in response to CTB-Pro-IGF-1 compared to commercial IGF-1. This may be due to activation of multiple pathways by internalization of CTB-Pro-IGF-1 binding to GM1 receptors in addition to IGF-1/IGF-1R interaction at the cell surface. Alternatively, the commercial preparations of IGF-1 do not contain the e-peptide, present in CTB-Pro-IGF-1 that might enhance the IGF-1 signaling pathway (Philippou et al., 2014). IGF-1 is involved in multiple growth signaling pathways including bone growth resulted from proliferation and differentiation of chondrogenic and osteogenic cells (Bikle et al., 2015). We found that CTB-Pro-IGF-1 not only promotes osteoblast proliferation but also significantly upregulates osteoblast marker gene expression including ALP, OSX, and RUNX2, demonstrating the efficacy of CTB-Pro-IGF-1 on osteogenesis. This study advances the therapeutic capacity of chloroplast derived Pro-IGF-1 for dental treatment based on existing evidence: proliferation of osteoblasts enhanced bone formation and regeneration, transplanted GMSCs resulted in regeneration of impaired periodontal tissue and differentiation to osteoblasts, and human oral epithelial growth was stimulated by keratinocyte growth factors that regulate keratinocyte formation and differentiation. Recent clinical trials have shown that application of recombinant human fibroblast (FGF)-2 and teriparatide (parathyroid 1-34) resulted in bone formation after they were delivered to the periodontal lesion via open flap debridement surgery (Rios et al., 2015). However, despite the promising emerging technology, surgical delivery is still required for delivering therapeutic proteins in dental medicine. The chloroplast system offers a platform for a novel periodontal drug delivery to increase patient compliance and affordability.

Fracture repair is a highly complex and dynamic physiological process, which involves characteristic phases of inflammation, bony callus formation and bone remodeling (Marsell et al., 2011). Several biological growth factors, such as TGF-β, BMP, PDGF, FGF and VEGF, have been shown to influence the process of bone healing by regulating cellular proliferation, migration, differentiation and other cellular processes (Devescovi et al., 2008). Among these growth factors, IGF-1 also shows a strong impact on bone regeneration in fracture healing. Both local and systemic administration of IGF-1 results in improved increased bone healing and rapid clinical improvements (Govoni et al., 2007; Jiang et al., 2006, Schmidmaier et al., 2002; and Locatelli et al., 2014). To examine the effect of orally delivered IGF-1 on bone regeneration in diabetic condition in vivo, we applied a diabetic fracture healing model in this study. We did not observe a significant impact on fracture healing after IGF-1 treatment in non-diabetic mice because endogenous IGF-1 levels were normal. Very interestingly, in the pathological diabetic condition, when the bone regeneration is reduced, IGF-1 can restore the defective bone regeneration caused by diabetes. We found the bone volume, density and area were significantly increased after IGF-1 treatment, when compared to the WT plant treated diabetic mice. It is worth noting that the blood glucose levels are not different between diabetic mice treated with or without IGF-1 during this study. This indicated that improved bone formation is due to its effect on the bone and not due to improving diabetic condition. The mechanisms involved in diabetes-induced skeletal impairment are not yet fully understood. It involves a series of molecular and cellular alterations resulting in imbalances in chondrocyte apoptosis, premature removal of cartilage, reduced osteoblast differentiation and function or alterations in vascularization. Further investigations are required to elucidate the mechanisms of IGF-1 treatment in diabetic bone.

In addition, we compared increases in IGF-1 in sera and muscle from PTD-Pro-IGF-1 and CTB-Pro-IGF-1 fed mice to distinguish efficiency of non-receptor mediated vs. receptor mediated delivery. We observed ˜28% increases of IGF-1 in muscle tissue for both PTD-Pro-IGF-1 and CTB-Pro-IGF-1 fed mice. In contrast to the receptor-mediated transport of drug to all types of cells by CTB, PTD penetrates cell membranes by stimulating macropinocytosis and does not enter immune modulatory cells (Xiao et al., 2016). The ability to deliver Pro-IGF1 to muscle tissue using PTD presents unique advantage in drug delivery for prevention of antibody development, which is often a challenge for several injected protein drugs in hemophilia or other diseases (Herzog et al., 2017 and Daniell et al., 2016).

In conclusion, this study demonstrates that the Pro-IGF-1 expressed in plant chloroplasts and bioencapsulated by the plant cell wall is orally deliverable in fully functional form. Lyophilized plant cells can be stored at room temperature for months without losing functional efficacy, facilitating affordability and enhanced patient compliance, in sharp contrast to surgical delivery of protein drugs in dental medicine. Precise excision of the antibiotic resistance gene from transformed lettuce chloroplast genome, inclusion of e-peptide to enhance function of IGF1 not present in current clinical products and ease of repetitive oral drug delivery offer major improvements for patients who suffer from various genetic muscle diseases, acute atrophy, injuries or bone loss.

### Example II

### Expression and Purification of Antibiotic Free Biopharmaceutical Proteins for Oral Consumption

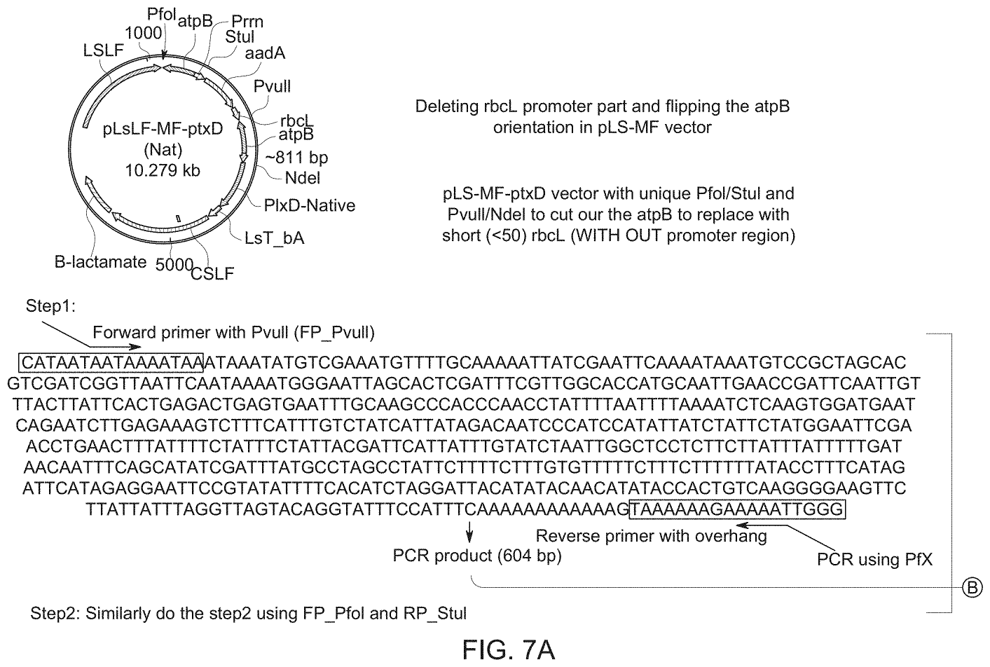

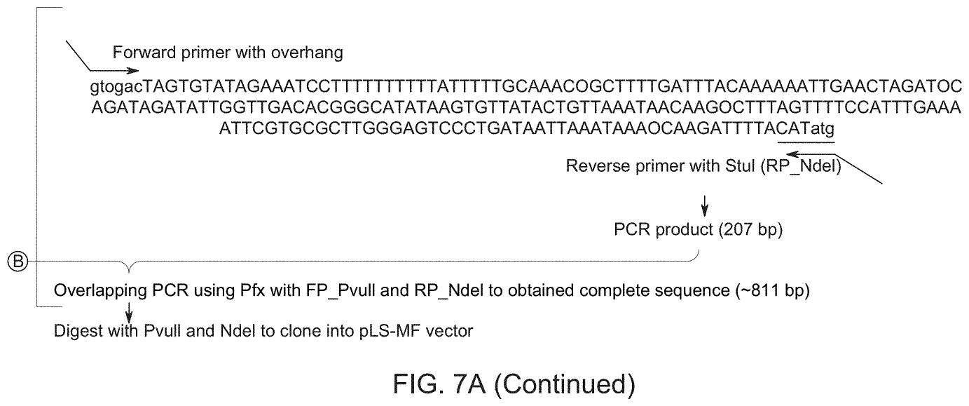

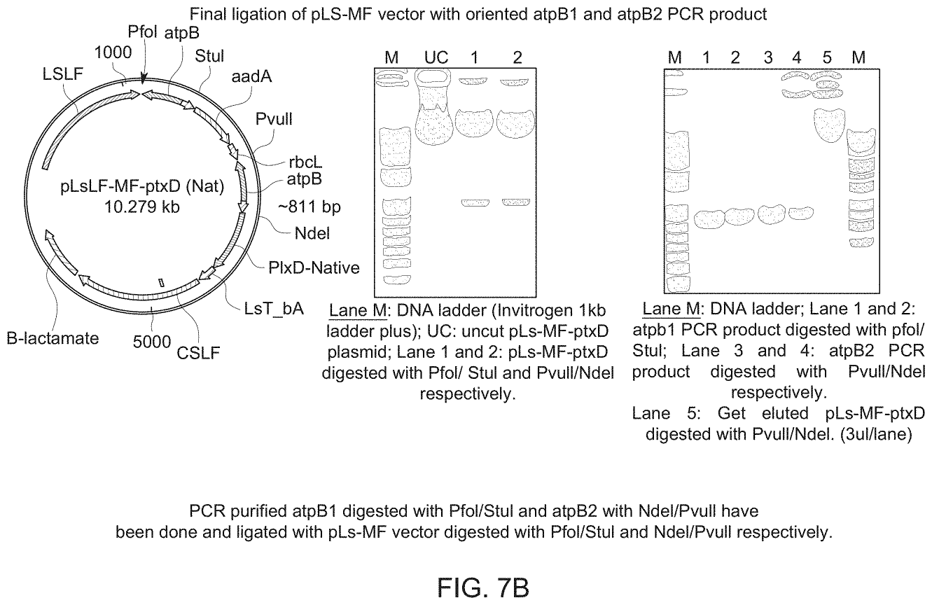

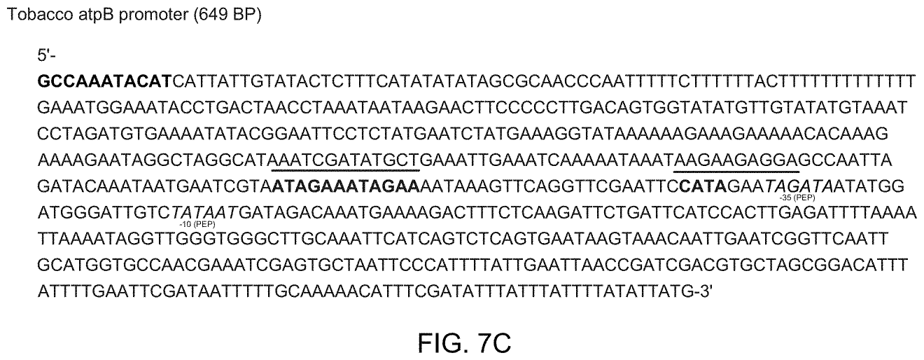

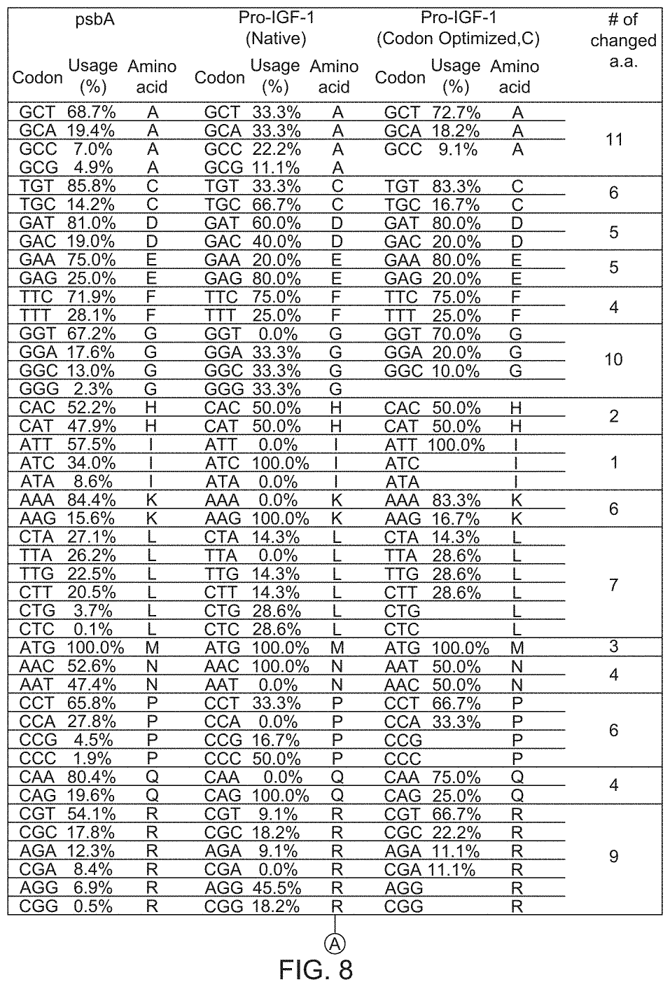

Example I provides a paradigm for producing biopharmaceuticals, as exemplified by the production of IGF-1, in the plastids of higher plants which are antibiotic free. FIG. 7A provides a brief schematic of the molecular cloning strategy used for producing marker free biopharmaceuticals in accordance with the present invention. FIG. 7B provides a schematic diagram of the final cloning vector. FIG. 7C provides the sequence of the 649 bp repeat described in the Example. The antibiotic selectable marker removal process includes the steps of deleting a portion of the rbcL promoter and flipping the orientation of atpB in a pLS-MF vector. As discussed in Example I, pLS-MF-ptxD vector contains unique PfoI/StuI and PvuII/NdeI sites for removing the atpB sequence which was then replaced with a short (<50) rbcL lacking the promoter region. While the 649 bp sequence repeat (SEQ ID NO: 16, FIG. 7C) was used in some experiments, this sequence was altered to remove certain promoter elements. The native atpB repeat sequence (649 bp) contains rbcL and atpB promoter regions in opposite directions and this disrupts gene expression by making antisense RNA when transgene cassette is introduced into the transcriptionally active spacer regions. However, such spacer regions are preferred because transgene expression level is higher than transcriptionally silent spacer regions (Jin & Daniell, Trends in Plant Science, 2015; Daniell et al, Genome Biology 2016). Therefore, one or both promoter regions were deleted in different versions of the marker-free expression cassette.

The successful expression of IGF-1 in antibiotic lettuce plants enables the expression of a variety of biopharmaceutical proteins, which include, but are not limited to those listed in the Table below.

| Biopharmaceutical | Marker-free vector | Reference for gene |
|---|---|---|
| proteins | designation | sequence |
| CTB or PTD Fusion | | |
| peptide genes | | |
| CTB-proinsulin | pLS-MF-Pris | PBJ 5: 495-510 |
| CTB-proinsulin | pLS-MF-Pris | PBJ 8:585-598 |
| CTB-proinsulin | pLS-MF-Pris | Plant Phys 152: 2088-2104 |
| CTB-AMA | pLS-MF-AMA | PBJ 8: 223-242 |
| CTB-MSP | pLS-MF-MSP1 | PBJ 8: 223-242 |
| CTB-FIX | | |
| With furin | pLS-MF-FFIX | PNAS 107: 7101 |
| Without furin | pLS-MF-FIX | PNAS 107: 7101 |
| CTB-FIX- furin | pLS-MF-FIX | Biomaterials 70: 84-93 |
| CTB-Exendin | pLS-MF-Ex4 | PBJ 11: 77-86 |
| CTB-ESAT6 | pLS-MF-ESAT6 | PLoS One 8: 54708 |
| CTB-Mtb72F | pLS-MF-Mtb72F | PLoS One 8: 54708 |
| CTB-VP1 | pLS-MF-VP1 | PBJ 14: 2190-2200 |
| CTB-MBP | pLS-MF-MBP | Mol. Ther 22: 535 |
| CTB-Ace 2 | pLS-MF-Ace2 | Hypertension 64: 1248 |
| CTB-Ang1-7 | pLS-MF-Ang1-7 | Mol Ther 22: 2069 |
| CTB-GAA | pLS-MF-GAA | PBJ 13: 1023-1032 |
| CTB-FVIII-HC | pLS-MF-FVIII-HC | Blood 124: 1659 |
| CTB-FVIII-C2 | pLS-MF-FVIII-C2 | Blood 124: 1659 |
| CTB-FVIII-HC (CO) | pLS-MF-FVIII-HC | Plant Phys 172: 62-77 |
| CTB-FVIII-LC (CO) | pLS-MF-FVIII-LC | PBJ 16: 1148-1160 |
| CTB-FVIII-SC (CO) | pLS-MF-FVIII-SC | PBJ 16: 1148-1160 |
| CTB-ProlGF1 | pLS-MF-ProlGF1 | |

In certain aspects, methods for treatment of subjects in need of IGF-1 therapy are also provided. An exemplary method entails oral administration of an effective amount of antibiotic free lettuce plants or lettuce plant products comprising IGF-1 protein operably linked to a PTD peptide and e-peptide which enhances IGF1 function, said administration being effective to alleviate or improve symptoms of genetic muscle diseases, acute atrophy, injuries or bone loss. While IGF-1 is exemplified herein, any of the proteins listed above can be expressed in marker free plants described herein. Such plants and plant products would have a variety of utilities for the treatment of diabetes, coagulation disorders, inborn errors of metabolism disorders, etc.

### REFERENCES

D. D. Bikle et al., Role of IGF-I signaling in muscle bone interactions. Bone 80, 79-88 (2015).
E. R. Barton, L. Morris, A. Musaro, N. Rosenthal, H. L. Sweeney, Muscle-specific expression of insulin-like growth factor I counters muscle decline in mdx mice. J Cell Biol 157, 137-148 (2002).
C. R. Heatwole et al., Open-label trial of recombinant human insulin-like growth factor 1/recombinant human insulin-like growth factor binding protein 3 in myotonic dystrophy type 1. Arch Neurol 68, 37-44 (2011).
K. E. Govoni et al., Conditional deletion of insulin-like growth factor-I in collagen type 1 alpha 2-expressing cells results in postnatal lethality and a dramatic reduction in bone accretion. Endocrinology 148, 5706-5715 (2007).
J. Jiang et al., Transgenic mice with osteoblast-targeted insulin-like growth factor-I show increased bone remodeling. Bone 39, 494-504 (2006).
G. Schmidmaier et al., Improvement of fracture healing by systemic administration of growth hormone and local application of insulin-like growth factor-1 and transforming growth factor-beta1. Bone 31, 165-172 (2002).
V. Locatelli, V. E. Bianchi, Effect of GH/IGF-1 on Bone Metabolism and Osteoporsosis. Int J Endocrinol 2014, 235060 (2014).
C. G. Tahimic, Y. Wang, D. D. Bikle, Anabolic effects of IGF-1 signaling on the skeleton. Front Endocrinol (Lausanne) 4, 6 (2013).
S. Wang et al., Insulin-like growth factor 1 can promote the osteogenic differentiation and osteogenesis of stem cells from apical papilla. Stem Cell Res 8, 346-356 (2012).
A. Philippou, M. Maridaki, S. Pneumaticos, M. Koutsilieris, The Complexity of the IGF1 Gene Splicing, Posttranslational Modification and Bioactivity. Molecular Medicine 20, 202-214 (2014).
J. Durzynska, A. Philippou, B. K. Brisson, M. Nguyen-McCarty, E. R. Barton, The pro-Forms of Insulin-Like Growth Factor I (IGF-I) Are Predominant in Skeletal Muscle and Alter IGF-I Receptor Activation. Endocrinology 154, 1215-1224 (2013).
A. Philippou, E. R. Barton, Optimizing IGF-I for skeletal muscle therapeutics. Growth Hormone & Igf Research 24, 157-163 (2014).
J. Su et al., Low cost industrial production of coagulation factor IX bioencapsulated in lettuce cells for oral tolerance induction in hemophilia B. Biomaterials 70, 84-93 (2015).
R. W. Herzog et al., Oral Tolerance Induction in Hemophilia B Dogs Fed with Transplastomic Lettuce. Molecular Therapy 25, 512-522 (2017).
H. Daniell, H. T. Chan, E. K. Pasoreck, Vaccination via Chloroplast Genetics: Affordable Protein Drugs for the Prevention and Treatment of Inherited or Infectious Human Diseases. Annu Rev Genet 50, 595-618 (2016).
K. C. Kwon, H. Daniell, Oral Delivery of Protein Drugs Bioencapsulated in Plant Cells. Molecular Therapy 24, 1342-1350 (2016).
H. Daniell, C. S. Lin, M. Yu, W. J. Chang, Chloroplast genomes: diversity, evolution, and applications in genetic engineering. Genome Biology 17, 134 (2016).
J. Bally et al., Plant physiological adaptations to the massive foreign protein synthesis occurring in recombinant chloroplasts. Plant Physiol 150, 1474-1481 (2009).
B. Zhang, B. Shanmugaraj, H. Daniell, Expression and functional evaluation of biopharmaceuticals made in plant chloroplasts. Current Opinion in Chemical Biology 38, 17-23 (2017).
R. Sanz-Barrio, A. F. Millan, P. Corral-Martinez, J. M. Segui-Simarro, I. Farran, Tobacco plastidial thioredoxins as modulators of recombinant protein production in transgenic chloroplasts. Plant Biotechnol J 9, 639-650 (2011).
S. Iamtham, A. Day, Removal of antibiotic resistance genes from transgenic tobacco plastids. Nature Biotechnology 18, 1172-1176 (2000).
H. Daniell et al., Validation of leaf and microbial pectinases: commercial launching of a new platform technology. Plant Biotechnology Journal 17, 1154-1166 (2019).
U. Kumari et al., Validation of leaf enzymes in the detergent and textile industries: launching of a new platform technology. Plant Biotechnology Journal 17, 1167-1182 (2019).
L. N. Queiroz et al., Evaluation of lettuce chloroplast and soybean cotyledon as platforms for production of functional bone morphogenetic protein 2. Transgenic Research 28, 213-224 (2019).
K. C. Kwon et al., Codon Optimization to Enhance Expression Yields Insights into Chloroplast Translation. Plant Physiology 172, 62-77 (2016).
S. J. Duguay, L. Z. Jie, D. F. Steiner, MUTATIONAL ANALYSIS OF THE INSULIN-LIKE GROWTH-FACTOR-I PROHORMONE PROCESSING SITE. Journal of Biological Chemistry 270, 17566-17574 (1995).
J. Sanchez, J. Holmgren, Cholera toxin structure, gene regulation and pathophysiological and immunological aspects. Cellular and Molecular Life Sciences 65, 1347-1360 (2008).
J. Xu, N. Zhang, On the way to commercializing plant cell culture platform for biopharmaceuticals: present status and prospect. Pharmaceutical bioprocessing 2, 499-518 (2014).
V. Devescovi, E. Leonardi, G. Ciapetti, E. Cenni, Growth factors in bone repair. La Chirurgia degli organi di movimento 92, 161-168 (2008).
Taliglucerase (Elelyso) for Gaucher Disease. Medical Letter on Drugs and Therapeutics 54, 56-56 (2012).
Y. Shaaltiel et al., Plant-based oral delivery of beta-glucocerebrosidase as an enzyme replacement therapy for Gaucher's disease. Plant Biotechnology Journal 13, 1033-1040 (2015).
S. A. Tilles, D. Petroni, FDA-approved peanut allergy treatment The first wave is about to crest. Annals of Allergy Asthma & Immunology 121, 145-149 (2018).
D. G. Burrin, T. J. Wester, T. A. Davis, S. Amick, J. P. Heath, Orally administered IGF-I
D. Fouque, S. C. Peng, E. Shamir, J. D. Kopple, Recombinant human insulin-like growth factor-1 induces an anabolic response in malnourished CAPD patients. Kidney International 57, 646-654 (2000).
G. Mountzios et al., Insulin-like growth factor 1 receptor (IGF1R) expression and survival in operable squamous-cell laryngeal cancer. PLoS One 8, e54048 (2013).
H. F. Rios et al., Emerging Regenerative Approaches for Periodontal Reconstruction: Practical Applications From the AAP Regeneration Workshop. Clinical Advances in Periodontics 5, 40-46 (2015).
R. Marsell, T. A. Einhorn, The biology of fracture healing. Injury 42, 551-555 (2011).
Y. H. Xiao et al., Low cost delivery of proteins bioencapsulated in plant cells to human non-immune or immune modulatory cells. Biomaterials 80, 68-79 (2016).
D. Verma, N. P. Samson, V. Koya, H. Daniell, A protocol for expression of foreign genes in chloroplasts. Nat Protoc 3, 739-758 (2008).
T. Ruhlman, D. Verma, N. Samson, H. Daniell, The Role of Heterologous Chloroplast Sequence Elements in Transgene Integration and Expression. Plant Physiology 152, 2088-2104 (2010).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An antibiotic free transplastomic plant expressing a biopharmaceutical protein in plastids, said proteins being selected from the group consisting of CTB-proIGF1, PTD-proIGF1, CTB-proinsulin, CTB-AMA, CTB-MSP, CTB-FIX, CTB-FIX-furin, CTB-Exendin, CTB-ESAT6, CTB-Mtb72F, CTB-VP1, CTB-MBP, CTB-Ace 2, CTB-Ang1-7, CTB-GAA, CTB-FVIII-HC, CTB-FVIII-C2, codon optimized CTB-FVIII-HC, codon optimized CTB-FVIII-LC, and codon optimized CTB-FVIII-SC.

2. The plant of claim 1, wherein said protein is selected from CTB-proIGF1 and PTD-proIGF1, each of said proteins containing an e-peptide.

3. The plant of claim 2, wherein said IGF1 amino acid sequence has been altered to remove glycosylation sites.

4. The plant of claim 1, wherein said protein is encoded by a nucleic acid which is codon optimized for plastid expression.

5. An isolated plant cell obtained from the plant of claim 2.

6. A method for producing plants producing antibiotic free biopharmaceuticals for oral consumption in plastids of higher plants, comprising,
a) introducing a plastid transformation vector into a plant cell, said vector comprising a selectable marker encoding an antibiotic, operably linked to a plastid promoter, said gene and promoter being flanked by directly repeated DNA sequences between 600-800 nucleotides in length, said vector further comprising a heterologous nucleic acid comprising a second plastid promoter operably linked to a nucleic acid sequence encoding a biopharmaceutical protein of interest, wherein said protein of interest optionally includes a targeting peptide and, or, said protein encoding nucleic acid is codon optimized for expression in said plant;
b) culturing plant cells of step a) in the presence of said antibiotic in a regeneration media for a suitable period for shoot production to occur;
c) assessing said shoots for selectable marker gene excision,
d) transferring shoots which exhibit selectable marker gene excision to antibiotic free media suitable for inducing root growth,
e) generating a transplastomic plant expressing said protein of interest which lacks said selectable marker gene, from roots induced in step d).

7. The method of claim 6, wherein plants of step e) are assessed for homoplasmy and said selectable marker gene is an aadA gene and said antibiotic is spectinomycin.

8. (canceled)

9. The method of claim 6, wherein said direct repeats are encoded by SEQ ID NO: 16.

10. The method of claim 6, wherein said heterologous nucleic acid encodes codon optimized IGF1 operably linked to a PTD peptide and an e-peptide.

11. The method of claim 6, wherein said heterologous nucleic acid encodes codon optimized IGF1 operably linked to a CTB peptide.

12. The method of claim 6, wherein said heterologous protein of interest is selected from CTB-proinsulin, CTB-AMA, CTB-MSP, CTB-FIX, CTB-FIX-furin, CTB-Exendin, CTB-ESAT6, CTB-Mtb72F, CTB-VP1, CTB-MBP, CTB-Ace 2, CTB-Ang1-7, CTB-GAA, CTB-FVIII-HC, CTB-FVIII-C2, codon optimized CTB-FVIII-HC, codon optimized CTB-FVIII-LC, and codon optimized CTB-FVIII-SC.

13. The method of claim 6, wherein said plant is selected from a lettuce, tomato, carrot and soybean.

14. (canceled)

15. The method of claim 6, wherein said time period in step b) is between 4 to 6 weeks.

16. (canceled)

17. The method of claim 6, wherein expression of said heterologous protein of interest is maintained in subsequent generations.

18. The method of claim 6, wherein said plants are assessed for marker excision using PCR with primers that detect the presence or absence of excision products.

19. The method of claim 6, wherein said directly repeated DNA sequences are from the atpB gene and lack one or more endogenous promoter elements shown in FIG. 6.

20. The method of claim 6, wherein said second promoter comprises a psbA promoter and 5′UTR endogenous to the plant species to be transformed.

21. The method of claim 6, comprising isolating said biopharmaceutical protein from the plant of step e).

22. A method for treatment of a subject in need of IGF-1 therapy comprising oral administration of an effective amount of an antibiotic-free lettuce plant or lettuce plant product comprising IGF-1 protein operably linked to a PTD peptide and an e-peptide, said e-peptide enhancing IGF-1 function, said administration being effective to alleviate or improve symptoms of genetic muscle diseases, acute atrophy, injuries, or bone loss or fracture and said administration results in increased levels IGF-1 in one or more of sera, muscle, or bone.

23. (canceled)

24. The method of claim 22, wherein administration results in osteoblast proliferation and/or differentiation as indicated by increased expression levels of ALP, Osx, and/or RUNX2.

25. (canceled)

26. A plant plastid transformation vector for use in the method of claim 22, comprising a selectable marker gene encoding an antibiotic operably linked to a plastid promoter, said gene and promoter being flanked by directly repeated DNA sequences between 600-800 nucleotides in length, said vector further comprising a heterologous nucleic acid comprising a second plastid promoter operably linked to a nucleic acid sequence encoding a biopharmaceutical protein of interest, wherein said protein of interest optionally includes a targeting peptide and, or, said protein encoding nucleic acid is codon optimized for expression in said plant, wherein said heterologous nucleic acid is codon optimized IGF-1 operably lined to PTD and e-peptide encoding nucleic acid sequences.

27. (canceled)
